(19) United States
(12) Patent Application Publication
(10) Pub. No.: US 2009/0124650 A1
(43) Pub. Date: May 14, 2009

(54) Method of Treating Pain Utilizing Controlled Release Oxymorphone Pharmaceutical Compositions and Instructions on Effects of Alcohol

(75) Inventor: Harry Ahdieh, Lincoln University, PA (US)

Correspondence Address: MAYER BROWN LLP, P.O. BOX 2828, CHICAGO, IL, 60690, US

(73) Assignee: Endo Pharmaceuticals, Inc., Chadds Ford, PA (US)

(21) Appl. No.: 11/766,748

(22) Filed: Jun. 21, 2007

(51) Int. Cl. A61K 31/485

(52) U.S. Cl. 514/282

(57) ABSTRACT

The invention pertains to a method of using oxymorphone in the treatment of pain by providing a patient with an oxymorphone dosage form and informing the patient or prescribing physician of the effect of alcohol on the maximum concentration of oxymorphone.

### BACKGROUND OF THE INVENTION

Pain is the most frequently reported symptom and it is a common clinical problem which confronts the clinician. Many millions of people in the USA suffer from severe pain that, according to numerous recent reports, is chronically undertreated or inappropriately managed. The clinical usefulness of the analgesic properties of opioids has been recognized for centuries, and morphine and its derivatives have been widely employed for analgesia for decades in a variety of clinical pain states.

Oxymorphone HCl (14-hydroxydihydromorphinone hydrochloride) is a semi-synthetic phenanthrene-derivative opioid agonist, widely used in the treatment of acute and chronic pain, with analgesic efficacy comparable to other opioid analgesics. Oxymorphone is currently marketed as an injection (1 mg/ml in 1 ml ampules; 1.5 mg/ml in 1 ml ampules; 1.5 mg/ml in 10 ml multiple dose vials) for intramuscular, subcutaneous, and intravenous administration, and as 5 mg rectal suppositories. At one time, 2 mg, 5 mg and 10 mg oral immediate release (IR) tablet formulations of oxymorphone HCl were marketed. Oxymorphone HCl is metabolized principally in the liver and undergoes conjugation with glucuronic acid and reduction to 6-alpha- and beta-hydroxy epimers.

An important goal of analgesic therapy is to achieve continuous relief of chronic pain. Regular administration of an analgesic is generally required to ensure that the next dose is given before the effects of the previous dose have worn off. Compliance with opioids increases as the required dosing frequency decreases. Non-compliance results in suboptimal pain control and poor quality of life outcomes. (Ferrell B et al. Effects of controlled-release morphine on quality of life for cancer pain. Oncol. Nur. Forum 1989; 4:521-26). Scheduled, rather than “as needed” administration of opioids is currently recommended in guidelines for their use in chronic non-malignant pain. Unfortunately, evidence from prior clinical trials and clinical experience suggests that the short duration of action of immediate release oxymorphone would necessitate administration every 4-6 hours in order to maintain optimal levels of analgesia in chronic pain. A controlled release formulation which would allow less frequent dosing of oxymorphone would be useful in pain management.

For instance, a controlled release formulation of morphine has been demonstrated to provide patients fewer interruptions in sleep, reduced dependence on caregivers, improved compliance, enhanced quality of life outcomes, and increased control over the management of pain. In addition, the controlled release formulation of morphine was reported to provide more constant plasma concentration and clinical effects, less frequent peak to trough fluctuations, reduced dosing frequency, and possibly fewer side effects. (Thirlwell M P et al., Pharmacokinetics and clinical efficacy of oral morphine solution and controlled-release morphine tablets in cancer patients. Cancer 1989; 63:2275-83; Goughnour B R et al., Analgesic response to single and multiple doses of controlled-release morphine tablets and morphine oral solution in cancer patients. Cancer 1989; 63:2294-97; Ferrell B. et al., Effects of controlled-release morphine on quality of life for cancer pain. Oncol. Nur. Forum 1989; 4:521-26.

There are two factors associated with the metabolism of some drugs that may present problems for their use in controlled release systems. One is the ability of the drug to induce or inhibit enzyme synthesis, which may result in a fluctuating drug blood plasma level with chronic dosing. The other is a fluctuating drug blood level due to intestinal (or other tissue) metabolism or through a hepatic first-pass effect.

Oxymorphone is metabolized principally in the liver, resulting in an oral bioavailability of about 10%. Evidence from clinical experience suggests that the short duration of action of immediate release oxymorphone necessitates a four hour dosing schedule to maintain optimal levels of analgesia. It would be useful to clinicians and patients alike to have controlled release dosage forms of oxymorphone to use to treat pain and a method of treating pain using the dosage forms.

A general dictum of opioid therapy, including oxymorphone therapy, states that ethanol should not be consumed at any time while receiving opioid therapy because of the known additive pharmacodynamic effects of ethanol and opioids (CNS and respiratory depression). Recently, however, information (both in vitro and in vivo) has emerged with some products (Palladone® and Avinza®) indicating that direct exposure to ethanol causes a disintegration of the ER formulations, which results in the rapid release of drug referred to as “dose dumping”.

### SUMMARY OF THE INVENTION

One aspect of the invention is a method of using oxymorphone in the treatment of pain comprising providing a patient with a therapeutically effective amount of oxymorphone in an oral dosage form and informing the patient or the patient's prescribing physician that the Cₘₐₓ of oxymorphone increased on average by about 70%, and up to about 270% in individual subjects, following concomitant administration with 240 mL of 40% ethanol.

Another aspect of the invention provides a method of using oxymorphone in the treatment of pain comprising providing a patient with a therapeutically effective amount of oxymorphone in an oral dosage form, informing the patient or the patient's prescribing physician that the Cₘₐₓ of oxymorphone increased on average by about 70%, and up to about 270% in individual subjects, following concomitant administration with 240 mL of 40% ethanol, and informing the patient or physician that the patient should not consume alcoholic beverages or medications containing alcohol when using the extended release oral dosage form of oxymorphone.

A further aspect of the invention provides a method of using oxymorphone in the treatment of pain comprising providing a patient with a therapeutically effective amount of oxymorphone in an oral dosage form, informing the patient or the patient's prescribing physician that the Cₘₐₓ of oxymorphone increased on average by about 70%, and up to about 270% in individual subjects, following concomitant administration with 240 mL of 40% ethanol, informing the patient or the patient's prescribing physician that the bioavailability of oxymorphone may be increased in patients with hepatic impairment, and informing the patient or the patient's prescribing physician that the bioavailability of oxymorphone is increased in patients with renal impairment.

### BRIEF DESCRIPTION OF THE FIGURES

FIG. 1 is a pharmacokinetic profile for 6-hydroxy oxymorphone with PID scores.

FIG. 2 is a pharmacokinetic profile for oxymorphone with PID scores.

FIG. 3 is a pharmacokinetic profile for 6-hydroxy oxymorphone with categorical pain scores.

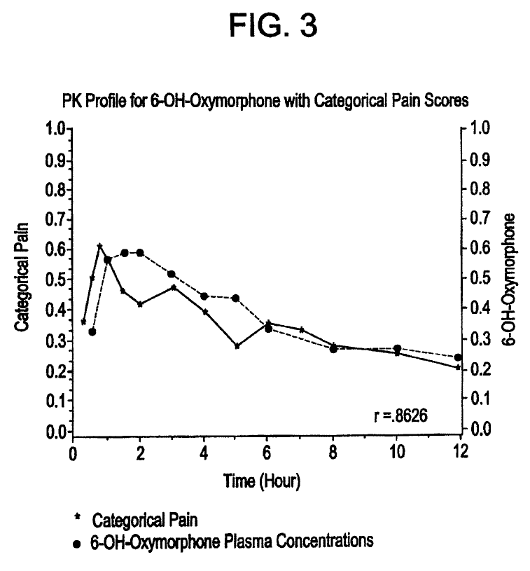

FIG. 4 is a pharmacokinetic profile for oxymorphone with categorical pain scores.

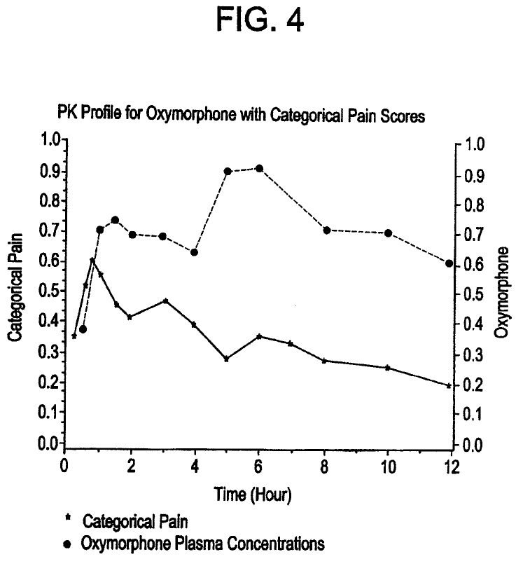

FIG. 5 is a graph of the mean blood plasma concentration of oxymorphone versus time for clinical study 1.

FIG. 6 is a graph of the mean blood plasma concentration of oxymorphone versus time for clinical study 2.

FIG. 7 is a graph of the mean blood plasma concentration of oxymorphone versus time for clinical study 3.

FIG. 8 is a graph of the mean blood plasma concentration of 6-hydroxy oxymorphone versus time for clinical study 3.

FIG. 9 is a graph of the mean blood plasma concentration of oxymorphone for immediate and controlled release tablets from a single dose study.

FIG. 10 is a graph of the mean blood plasma concentration of oxymorphone for immediate and controlled release tablets from a steady state study.

FIG. 11 is a graph of the dissolution rofiles of 40 mg oxymorphone HCl ER tablets in 0.1N HCl and ethanol solutions.

FIG. 12 is a graph of the mean oxymorphone plasma concentration-time curves for all treatments (treatments excluding subjects who vomited).

FIG. 13 is a graph of the cumulative Cₘₐₓ ratios by treatment (treatments excluding subjects who vomited).

FIG. 14 is a graph of the mean oxymorphone plasma concentration-time curves for all subjects (n=25).

FIG. 15 is a graph of the cumulative Cₘₐₓ ratios.

### DETAILED DESCRIPTION OF THE INVENTION

The present invention provides methods of using oxymorphone in the treatment of pain. In one aspect of the invention the method may involve steps of providing a patient with a therapeutically effective amount of oxymorphone in an extended release oral dosage form and informing the patient or the patient's prescribing physician that the Cₘₐₓ of oxymorphone increased on average by about 70%, and up to about 270% in individual subjects, following concomitant administration with 240 mL of 40% ethanol.

Among the controlled (or extended) release, as well as immediate release, pharmaceutical compounds comprising oxymorphone that may be used in the methods of this invention is Opana®, which upon its approval on Jun. 22, 2006 became the first-ever controlled release oxymorphone formulation to be approved by the United States Food and Drug Administration (FDA). Opana® is available in both immediate release and controlled or extended release dosage forms. The approved labels of Opana® are incorporated herein by reference to the extent permitted by law.

The present invention also provides methods for alleviating pain for 12 to 24 hours using a single dose of a pharmaceutical composition by producing a blood plasma level of oxymorphone and/or 6-OH oxymorphone of at least a minimum value for at least 12 hours or more. As used herein, the terms “6-OH oxymorphone” and “6-hydroxy oxymorphone” are interchangeable and refer to the analog of oxymorphone having an alcohol (hydroxy) moiety that replaces the carboxy moiety found on oxymorphone at the 6-position.

To overcome the difficulties associated with a 4-6 hourly dosing frequency of oxymorphone, this invention provides an oxymorphone controlled release oral solid dosage form, comprising a therapeutically effective amount of oxymorphone or a pharmaceutically acceptable salt of oxymorphone. It has been found that the decreased rate of release of oxymorphone from the oral controlled release formulation of this invention does not substantially decrease the bioavailability of the drug as compared to the same dose of a solution of oxymorphone administered orally. The bioavailability is sufficiently high and the release rate is such that a sufficient plasma level of oxymorphone and/or 6-OH oxymorphone is maintained to allow the controlled release dosage to be used to treat patients suffering moderate to severe pain with once or twice daily dosing. The dosing form of the present invention can also be used with thrice daily dosing.

It is critical when considering the present invention that the difference between a controlled release tablet and an immediate release formulation be fully understood. In classical terms, an immediate release formulation releases at least 80% of its active pharmaceutical ingredient within 30 minutes. With reference to the present invention, the definition of an immediate release formulation will be broadened further to include a formulation which releases more than about 80% of its active pharmaceutical ingredient within 60 minutes in a standard USP Paddle Method dissolution test at 50 rpm in 500 ml media having a pH of between 1.2 and 6.8 at 37° C. “Controlled release” formulations, as referred to herein, will then encompass any formulations which release no more than about 80% of their active pharmaceutical ingredients within 60 minutes under the same conditions.

The controlled release dosage form of this invention exhibits a dissolution rate in vitro, when measured by USP Paddle Method at 50 rpm in 500 ml media having a pH between 1.2 and 6.8 at 37° C., of about 15% to about 50% by weight oxymorphone released after 1 hour, about 45% to about 80% by weight oxymorphone released after 4 hours, and at least about 80% by weight oxymorphone released after 10 hours.

When administered orally to humans, an effective controlled release dosage form of oxymorphone should exhibit the following in vivo characteristics: (a) peak plasma level of oxymorphone occurs within about 1 to about 8 hours after administration; (b) peak plasma level of 6-OH oxymorphone occurs within about 1 to about 8 hours after administration; (c) duration of analgesic effect is through about 8 to about 24 hours after administration; (d) relative oxymorphone bioavailability is in the range of about 0.5 to about 1.5 compared to an orally-administered aqueous solution of oxymorphone; and (e) the ratio of the area under the curve of blood plasma level vs. time for 6-OH oxymorphone compared to oxymorphone is in the range of about 0.5 to about 1.5. Of course, there is variation of these parameters among subjects, depending on the size and weight of the individual subject, the subject's age, individual metabolism differences, and other factors. Indeed, the parameters may vary in an individual from day to day. Accordingly, the parameters set forth above are intended to be mean values from a sufficiently large study so as to minimize the effect of individual variation in arriving at the values. A convenient method for arriving at such values is by conducting a study in accordance with standard FDA procedures such as those employed in producing results for use in a new drug application (or abbreviated new drug application) before the FDA. Any reference to mean values herein, in conjunction with desired results, refer to results from such a study, or some comparable study. Reference to mean values reported herein for studies actually conducted are arrived at using standard statistical methods as would be employed by one skilled in the art of pharmaceutical formulation and testing for regulatory approval.

In one specific embodiment of the controlled release matrix form of the invention, the oxymorphone or salt of oxymorphone is dispersed in a controlled release delivery system that comprises a hydrophilic material which, upon exposure to gastrointestinal fluid, forms a gel matrix that releases oxymorphone at a controlled rate. The rate of release of oxymorphone from the matrix depends on the drug's partition coefficient between components of the matrix and the aqueous phase within the gastrointestinal tract. In a preferred form of this embodiment, the hydrophilic material of the controlled release delivery system comprises a mixture of a heteropolysaccharide gum and an agent capable of cross-linking the heteropolysaccharide in presence of gastrointestinal fluid. The controlled release delivery system may also comprise a water-soluble pharmaceutical diluent mixed with the hydrophilic material. Preferably, the cross-linking agent is a homopolysaccharide gum and the inert pharmaceutical diluent is a monosaccharide, a disaccharide, or a polyhydric alcohol, or a mixture thereof.

In a specific preferred embodiment, the appropriate blood plasma levels of oxymorphone and 6-hydroxy oxymorphone are achieved using oxymorphone in the form of oxymorphone hydrochloride, wherein the weight ratio of heteropolysaccharide to homopolysaccharide is in the range of about 1:3 to about 3:1, the weight ratio of heteropolysaccharide to diluent is in the range of about 1:8 to about 8:1, and the weight ratio of heteropolysaccharide to oxymorphone hydrochloride is in the range of about 10:1 to about 1:10. A preferred heteropolysaccharide is xanthan gum and a preferred homopolysaccharide is locust bean gum. The dosage form also comprises a cationic cross-linking agent and a hydrophobic polymer. In the preferred embodiment, the dosage form is a tablet containing about 5 mg to about 80 mg of oxymorphone hydrochloride. In a most preferred embodiment, the tablet contains about 20 mg oxymorphone hydrochloride.

The invention includes a method which comprises achieving appropriate blood plasma levels of drug while providing extended pain relief by administering one to three times per day to a patient suffering moderate to severe, acute or chronic pain, an oxymorphone controlled release oral solid dosage form of the invention in an amount sufficient to alleviate the pain for a period of about 8 hours to about 24 hours. This type and intensity of pain is often associated with cancer, autoimmune diseases, infections, surgical and accidental traumas and osteoarthritis.

The invention also includes a method of making an oxymorphone controlled release oral solid dosage form of the invention which comprises mixing particles of oxymorphone or a pharmaceutically acceptable salt of oxymorphone with granules comprising the controlled release delivery system, preferably followed by directly compressing the mixture to form tablets.

Pharmaceutically acceptable salts of oxymorphone which can be used in this invention include salts with the inorganic and organic acids which are commonly used to produce nontoxic salts of medicinal agents. Illustrative examples would be those salts formed by mixing oxymorphone with hydrochloric, sulfuric, nitric, phosphoric, phosphorous, hydrobromic, maleric, malic, ascorbic, citric or tartaric, pamoic, lauric, stearic, palmitic, oleic, myristic, lauryl sulfuric, naphthylenesulfonic, linoleic or linolenic acid, and the like. The hydrochloride salt is preferred.

It has now been found that 6-OH oxymorphone, which is one of the metabolites of oxymorphone, may play a role in alleviating pain. When oxymorphone is ingested, part of the dosage gets into the bloodstream to provide pain relief, while another part is metabolized to 6-OH oxymorphone. This metabolite then enters the bloodstream to provide further pain relief. Thus it is believed that both the oxymorphone and 6-hydroxyoxymorphone levels are important to pain relief.

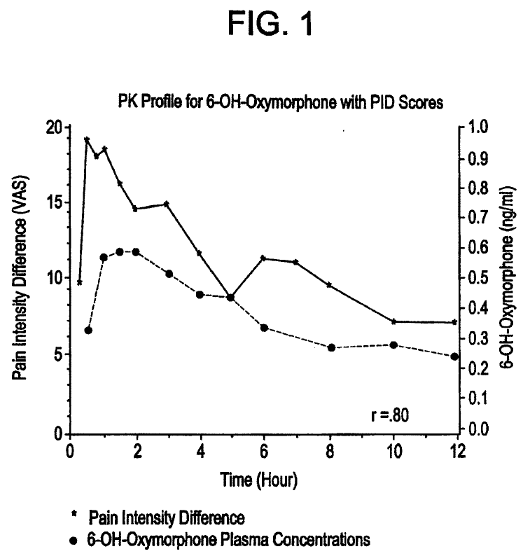

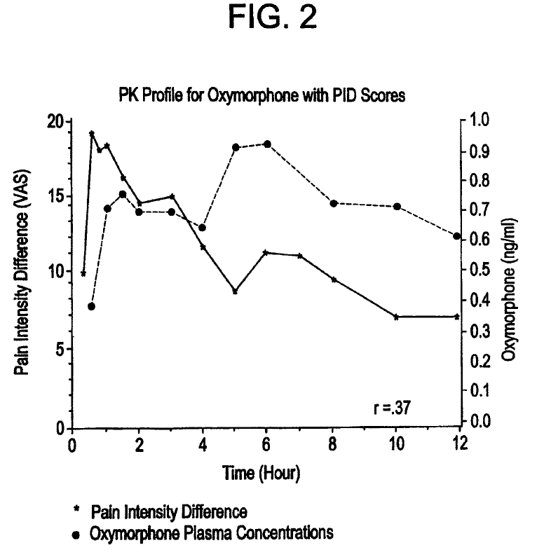

The effectiveness of oxymorphone and 6-hydroxyoxymorphone at relieving pain and the pharmacokinetics of a single dose of oxymorphone were studied. The blood plasma levels of both oxymorphone and 6-hydroxyoxymorphone were measured in patients after a single dose of oxymorphone was administered. Similarly, the pain levels in patients were measured after a single administration of oxymorphone to determine the effective duration of pain relief from a single dose. FIGS. 1-2 show the results of these tests, comparing pain levels to oxymorphone and 6-hydroxy oxymorphone levels.

For these tests, pain was measured using a Visual Analog Scale (VAS) or a Categorical Scale. The VAS scales consisted of a horizontal line, 100 mm in length. The left-hand end of the scale (0 mm) was marked with the descriptor “No Pain” and the right-hand end of the scale (100 mm) was marked with the descriptor “Extreme Pain”. Patients indicated their level of pain by making a vertical mark on the line. The VAS score was equal to the distance (in mm) from the left-hand end of the scale to the patient's mark. For the categorical scale, patients completed the following statement, “My pain at this time is” using the scale None=0, Mild=1, Moderate=2, or Severe=3.

As can be seen from these figures, there is a correlation between pain relief and both oxymorphone and 6-hydroxyoxymorphone levels. As the blood plasma levels of oxymorphone and 6-hydroxyoxymorphone increase, pain decreases (and pain intensity difference and pain relief increases). Thus, to the patient, it is the level of oxymorphone and 6-hydroxyoxymorphone in the blood plasma which is most important. Further it is these levels which dictate the efficacy of the dosage form. A dosage form which maintains a sufficiently high level of oxymorphone or 6-hydroxyoxymorphone for a longer period need not be administered frequently. Such a result is accomplished by embodiments of the present invention.

The oxymorphone controlled release oral solid dosage form of this invention can be made using any of several different techniques for producing controlled release oral solid dosage forms of opioid analgesics.

In one embodiment, a core comprising oxymorphone or oxymorphone salt is coated with a controlled release film which comprises a water insoluble material and which upon exposure to gastrointestinal fluid releases oxymorphone from the core at a controlled rate. In a second embodiment, the oxymorphone or oxymorphone salt is dispersed in a controlled release delivery system that comprises a hydrophilic material which upon exposure to gastrointestinal fluid forms a gel matrix that releases oxymorphone at a controlled rate. A third embodiment is a combination of the first two: a controlled release matrix coated with a controlled release film. In a fourth embodiment the oxymorphone is incorporated into an osmotic pump. In any of these embodiments, the dosage form can be a tablet, a plurality of granules in a capsule, or other suitable form, and can contain lubricants, colorants, diluents, and other conventional ingredients.

Osmotic Pump

An osmotic pump comprises a shell defining an interior compartment and having an outlet passing through the shell. The interior compartment contains the active pharmaceutical ingredient. Generally the active pharmaceutical ingredient is mixed with excipients or other compositions such as a polyalkylene. The shell is generally made, at least in part, from a material (such as cellulose acetate) permeable to the liquid of the environment where the pump will be used, usually stomach acid. Once ingested, the pump operates when liquid diffuses through the shell of the pump. The liquid dissolves the composition to produce a saturated situation. As more liquid diffuses into the pump, the saturated solution containing the pharmaceutical is expelled from the pump through the outlet. This produces a nearly constant release of active ingredient, in the present case, oxymorphone.

Controlled Release Coating

In this embodiment, a core comprising oxymorphone or oxymorphone salt is coated with a controlled release film which comprises a water insoluble material. The film can be applied by spraying an aqueous dispersion of the water insoluble material onto the core. Suitable water insoluble materials include alkyl celluloses, acrylic polymers, waxes (alone or in admixture with fatty alcohols), shellac and zein. The aqueous dispersions of alkyl celluloses and acrylic polymers preferably contain a plasticizer such as triethyl citrate, dibutyl phthalate, propylene glycol, and polyethylene glycol. The film coat can contain a water-soluble material such as polyvinylpyrrolidone (PVP) or hydroxypropylmethylcellulose (HPMC).

The core can be a granule made, for example, by wet granulation of mixed powders of oxymorphone or oxymorphone salt and a binding agent such as HPMC, or by coating an inert bead with oxymorphone or oxymorphone salt and a binding agent such as HPMC, or by spheronising mixed powders of oxymorphone or oxymorphone salt and a spheronising agent such as microcrystalline cellulose. The core can be a tablet made by compressing such granules or by compressing a powder comprising oxymorphone or oxymorphone salt.

The in vitro and in vivo release characteristics of this controlled release dosage form can be modified by using mixtures of different water insoluble and water soluble materials, using different plasticizers, varying the thickness of the controlled release film, including release-modifying agents in the coating, or by providing passageways through the coating.

Controlled Release Matrix

It is important in the present invention that appropriate blood plasma levels of oxymorphone and 6-hydroxy oxymorphone be achieved and maintained for sufficient time to provide pain relief to a patient for a period of 12 to 24 hours. The preferred composition for achieving and maintaining the proper blood plasma levels is a controlled-release matrix. In this embodiment, the oxymorphone or oxymorphone salt is dispersed in a controlled release delivery system that comprises a hydrophilic material (gelling agent) which upon exposure to gastrointestinal fluid forms a gel matrix that releases oxymorphone at a controlled rate. Such hydrophilic materials include gums, cellulose ethers, acrylic resins, and protein-derived materials. Suitable cellulose ethers include hydroxyalkyl celluloses and carboxyalkyl celluloses, especially hydroxyethyl cellulose (HEC), hydroxypropyl cellulose (HPC), HPMC, and carboxy methylcellulose (CMC). Suitable acrylic resins include polymers and copolymers of acrylic acid, methacrylic acid, methyl acrylate and methyl methacrylate. Suitable gums include heteropolysaccharide and homopolysaccharide gums, e.g., xanthan, tragacanth, acacia, karaya, alginates, agar, guar, hydroxypropyl guar, carrageenan, and locust bean gums.

Preferably, the controlled release tablet of the present invention is formed from (I) a hydrophilic material comprising (a) a heteropolysaccharide; or (b) a heteropolysaccharide and a cross-linking agent capable of cross-linking said heteropolysaccharide; or (c) a mixture of (a), (b) and a polysaccharide gum; and (II) an inert pharmaceutical filler comprising up to about 80% by weight of the tablet; and (III) oxymorphone.

The term “heteropolysaccharide” as used herein is defined as a water-soluble polysaccharide containing two or more kinds of sugar units, the heteropolysaccharide having a branched or helical configuration, and having excellent water-wicking properties and immense thickening properties.

A preferred heteropolysaccharide is xanthan gum, which is a high molecular weight (>10⁶) heteropolysaccharide. Other preferred heteropolysaccharides include derivatives of xanthan gum, such as deacylated xanthan gum, the carboxymethyl ether, and the propylene glycol ester.

The cross linking agents used in the controlled release embodiment of the present invention which are capable of cross-linking with the heteropolysaccharide include homopolysaccharide gums such as the galactomannans, i.e., polysaccharides which are composed solely of mannose and galactose. Galactomannans which have higher proportions of unsubstituted mannose regions have been found to achieve more interaction with the heteropolysaccharide. Locust bean gum, which has a higher ratio of mannose to the galactose, is especially preferred as compared to other galactomannans such as guar and hydroxypropyl guar.

Preferably, the ratio of heteropolysaccharide to homopolysaccharide is in the range of about 1:9 to about 9:1, preferably about 1:3 to about 3:1. Most preferably, the ratio of xanthan gum to polysaccharide material (i.e., locust bean gum, etc.) is preferably about 1:1.

In addition to the hydrophilic material, the controlled release delivery system can also contain an inert pharmaceutical diluent such as a monosaccharide, a disaccharide, a polyhydric alcohol and mixtures thereof. The ratio of diluent to hydrophilic matrix-forming material is generally in the range of about 1:3 to about 3:1.

The controlled release properties of the controlled release embodiment of the present invention may be optimized when the ratio of heteropolysaccharide gum to homopolysaccharide material is about 1:1, although heteropolysaccharide gum in an amount of from about 20 to about 80% or more by weight of the heterodisperse polysaccharide material provides an acceptable slow release product. The combination of any homopolysaccharide gums known to produce a synergistic effect when exposed to aqueous solutions may be used in accordance with the present invention. It is also possible that the type of synergism which is present with regard to the gum combination of the present invention could also occur between two homogeneous or two heteropolysaccharides. Other acceptable gelling agents which may be used in the present invention include those gelling agents well-known in the art. Examples include vegetable gums such as alginates, carrageenan, pectin, guar gum, xanthan gum, modified starch, hydroxypropylmethylcellulose, methylcellulose, and other cellulosic materials such as sodium carboxymethylcellulose and hydroxypropyl cellulose. This list is not meant to be exclusive.

The combination of xanthan gum with locust bean gum with or without the other homopolysaccharide gums is an especially preferred gelling agent. The chemistry of certain of the ingredients comprising the excipients of the present invention such as xanthan gum is such that the excipients are considered to be self-buffering agents which are substantially insensitive to the solubility of the medicament and likewise insensitive to the pH changes along the length of the gastrointestinal tract.

The inert filler of the sustained release excipient preferably comprises a pharmaceutically acceptable saccharide, including a monosaccharide, a disaccharide, or a polyhydric alcohol, and/or mixtures of any of the foregoing. Examples of suitable inert pharmaceutical fillers include sucrose, dextrose, lactose, microcrystalline cellulose, fructose, xylitol, sorbitol, mixtures thereof and the like. However, it is preferred that a soluble pharmaceutical filler such as lactose, dextrose, sucrose, or mixtures thereof be used.

The cationic cross-linking agent which is optionally used in conjunction with the controlled release embodiment of the present invention may be monovalent or multivalent metal cations. The preferred salts are the inorganic salts, including various alkali metal and/or alkaline earth metal sulfates, chlorides, borates, bromides, citrates, acetates, lactates, etc. Specific examples of suitable cationic cross-linking agents include calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate and sodium fluoride. Multivalent metal cations may also be utilized. However, the preferred cationic cross-linking agents are bivalent. Particularly preferred salts are calcium sulfate and sodium chloride. The cationic cross-linking agents of the present invention are added in an amount effective to obtain a desirable increased gel strength due to the cross-linking of the gelling agent (e.g., the heteropolysaccharide and homopolysaccharide gums). In preferred embodiments, the cationic cross-linking agent is included in the sustained release excipient of the present invention in an amount from about 1 to about 20% by weight of the sustained release excipient, and in an amount about 0.5% to about 16% by weight of the final dosage form.

In the controlled release embodiments of the present invention, the sustained release excipient comprises from about 10 to about 99% by weight of a gelling agent comprising a heteropolysaccharide gum and a homopolysaccharide gum, from about 1 to about 20% by weight of a cationic crosslinking agent, and from about 0 to about 89% by weight of an inert pharmaceutical diluent. In other embodiments, the sustained release excipient comprises from about 10 to about 75% gelling agent, from about 2 to about 15% cationic crosslinking agent, and from about 30 to about 75% inert diluent. In yet other embodiments, the sustained release excipient comprises from about 30 to about 75% gelling agent, from about 5 to about 10% cationic cross-linking agent, and from about 15 to about 65% inert diluent.

The sustained release excipient used in this embodiment of the present invention (with or without the optional cationic cross-linking agent) may be further modified by incorporation of a hydrophobic material which slows the hydration of the gums without disrupting the hydrophilic matrix. This is accomplished in preferred embodiments of the present invention by granulating the sustained release excipient with the solution or dispersion of a hydrophobic material prior to the incorporation of the medicament. The hydrophobic polymer may be selected from an alkylcellulose such as ethylcellulose, other hydrophobic cellulosic materials, polymers or copolymers derived from acrylic or methacrylic acid esters, copolymers of acrylic and methacrylic acid esters, zein, waxes, shellac, hydrogenated vegetable oils, and any other pharmaceutically acceptable hydrophobic material known to those skilled in the art. The amount of hydrophobic material incorporated into the sustained release excipient is that which is effective to slow the hydration of the gums without disrupting the hydrophilic matrix formed upon exposure to an environmental fluid. In certain preferred embodiments of the present invention, the hydrophobic material is included in the sustained release excipient in an amount from about 1 to about 20% by weight. The solvent for the hydrophobic material may be an aqueous or organic solvent, or mixtures thereof.

Examples of commercially available alkylcelluloses are Aquacoat coating (aqueous dispersion of ethylcellulose available from FMC of Philadelphia, Pa.) and Surelease coating (aqueous dispersion of ethylcellulose available from Colorcon of West Point, Pa.). Examples of commercially available acrylic polymers suitable for use as the hydrophobic material include Eudragit RS and RL polymers (copolymers of acrylic and methacrylic acid esters having a low content (e.g., 1:20 or 1:40) of quaternary ammonium compounds available from Rohm America of Piscataway, N.J.).

The controlled release matrix useful in the present invention may also contain a cationic cross-linking agent such as calcium sulfate in an amount sufficient to cross-link the gelling agent and increase the gel strength, and an inert hydrophobic material such as ethyl cellulose in an amount sufficient to slow the hydration of the hydrophilic material without disrupting it. Preferably, the controlled release delivery system is prepared as a pre-manufactured granulation.

It has now been found that consumption of alcohol with the administration of the controlled release delivery system comprising oxymorphone affects the release of oxymorphone from the matrix, but does not cause the “dose dumping” or extreme increases in bioavailability that are seen in other controlled release opioid drugs. However, it has been found that the Cₘₐₓ of oxymorphone following concomitant administration of ethanol (alcohol) is still increased somewhat, with a mean of about a 70% increase, up to about 270% in an individual patient, when given 240 mL of 40% alcohol.

As such, one embodiment of the invention includes informing the patient or the patient's prescribing physician of the Cₘₐₓ changes resultant from various levels of alcohol consumption. Another embodiment includes informing the patient or physician that the AUC is essentially unchanged (in the case of 240 mL of 20% or 4% ethanol administered concomitantly) or not statistically significantly changed (in the case of concomitant administration of 240 mL or 40% ethanol). A further aspect of the invention includes informing the patient or physician of the food effects on Cₘₐₓ of oxymorphone. The Cₘₐₓ is 70% greater at consumption of 240 mL of 40% ethanol than without while it is 50% greater with a meal than without. Thus, the effect of concomitant administration of 240 mL of 40% alcohol is about 40% greater than the food effect. With lower amounts of alcohol consumed, it has been found that the effect of alcohol is less than the food effect. It has also been found that in elderly patients (65 years old or older), there is an about 1.4 fold increase in bioavailability and an about 1.5 fold increase in Cₘₐₓ of oxymorphone. Thus, another embodiment comprises informing the patient or physician of such effects on bioavailability and Cₘₐₓ for elderly patients.

Another embodiment of the invention comprises informing the patient or physician about the additive effects of oxymorphone and alcohol. A further embodiment comprises informing the patient or physician that the patient should not consume alcohol with oxymorphone because additive effects may occur. Another embodiment comprises informing that patient or physician that the patient should not consumer alcoholic beverages or medications containing alcohol when using the extended release oral dosage form of oxymorphone.

The information can be communicated to the physician or patient in numerous ways that will be evident to one of skill in the art. For example, such instructions could be included in the labeling information, which can be for example the FDA-approved labeling, a package insert, or on the label itself. Other ways of communicating with patients or physicians are also available and are contemplated by the present invention.

It has also been found that the bioavailability of oxymorphone is also increased in patients who are hepatically impaired (i.e. have impaired liver function). Thus, in one embodiment the invention comprises informing the patient or the patient's prescribing physician that the bioavailability of oxymorphone may be increased in patients with hepatic impairment, as it is important that the patient and/or doctor be made aware of this effect. It has further been found that the bioavailability of oxymorphone is increased in patients who are renally impaired (i.e. have impaired kidney function). Thus, in another embodiment the invention comprises informing the patient or the patient's prescribing physician that the bioavailability of oxymorphone is increased in patients with renal impairment. Applicant hereby incorporates by reference in their entirety its United States patent applications titled “METHOD OF TREATING PAIN UTILIZING CONTROLLED RELEASE OXYMORPHONE PHARMACEUTICAL COMPOSITIONS AND INSTRUCTION ON DOSING FOR HEPATIC IMPAIRMENT” and “METHOD OF TREATING PAIN UTILIZING CONTROLLED RELEASE OXYMORPHONE PHARMACEUTICAL COMPOSITIONS AND INSTRUCTIONS ON DOSING FOR RENAL IMPAIRMENT”, filed concurrently herewith.

### EXAMPLES

### Example 1

Two controlled release delivery systems are prepared by dry blending xanthan gum, locust bean gum, calcium sulfate dehydrate, and dextrose in a high speed mixed/granulator for 3 minutes. A slurry is prepared by mixing ethyl cellulose with alcohol. While running choppers/impellers, the slurry is added to the dry blended mixture, and granulated for another 3 minutes. The granulation is then dried to a LOD (loss on drying) of less than about 10% by weight. The granulation is then milled using 20 mesh screen. The relative quantities of the ingredients are listed in the table below.

TABLE 1
Controlled Release Delivery System

| | Formulation 1 | Formulation 2 |
|---|---|---|
| Excipient | (%) | (%) |
| Locust Bean Gum, FCC | 25.0 | 30.0 |
| Xanthan Gum, NF | 25.0 | 30.0 |
| Dextrose, USP | 35.0 | 40.0 |
| Calcium Sulfate Dihydrate, NF | 10.0 | 0.0 |
| Ethylcellulose, NF | 5.0 | 0.0 |
| Alcohol, SD3A (Anhydrous) | (10)¹ | (20.0)¹ |
| Total | 100.0 | 100.0 |

A series of tablets containing different amounts of oxymorphone hydrochloride were prepared using the controlled release delivery Formulation 1 shown in Table 1. The quantities of ingredients per tablet are as listed in the following table.

TABLE 2
Sample Tablets of Differing Strengths

| Component | Amounts in Tablet (mg) | | | | |
|---|---|---|---|---|---|
| Oxymorphone HCl, | 5 | 10 | 20 | 40 | 80 |
| USP (mg) | | | | | |
| Controlled release | 160 | 160 | 160 | 160 | 160 |
| delivery system | | | | | |
| Silicified | 20 | 20 | 20 | 20 | 20 |
| microcrystalline | | | | | |
| cellulose, N.F. | | | | | |
| Sodium stearyl | 2 | 2 | 2 | 2 | 2 |
| fumarate, NF | | | | | |
| Total weight | 187 | 192 | 202 | 222 | 262 |
| Opadry (colored) | 7.48 | 7.68 | 8.08 | 8.88 | 10.48 |
| Opadry (clear) | 0.94 | 0.96 | 1.01 | 1.11 | 1.31 |

### Examples 2, 3 and 4

Two batches of 20 mg tablets were prepared as described above, using the controlled release delivery system of Formulation 1. One batch was formulated to provide relatively fast controlled release, the other batch was formulated to provide relatively slow controlled release. Compositions of the tablets are shown in the following table.

TABLE 3
Slow and Fast Release Compositions

| | Example 2 | Example 3 | Example 4 |
|---|---|---|---|
| Ingredients | Slow (mg) | Fast (mg) | Fast (mg) |
| Oxymorphone HCl, USP | 20 | 20 | 20 |
| Controlled Release Delivery System | 360 | 160 | 160 |
| Silicified Microcrystalline Cellulose, | 20 | 20 | 20 |
| NF | | | |
| Sodium stearyl fumarate, NF | 4 | 2 | 2 |
| Total weight | 404 | 202 | 202 |
| Coating (color or clear) | 12 | 12 | 9 |

The tablets of Examples 2, 3, and 4 were tested for in vitro release rate according to USP Procedure Drug Release U.S. Pat. No. 23. Release rate is a critical variable in attempting to control the blood plasma levels of oxymorphone and 6-hydroxyoxymorphone in a patient. Results are shown in the following Table 4.

TABLE 4
Release Rates of Slow and Fast Release Tablets

| Time | Example 2 | Example 3 | Example 4 |
|---|---|---|---|
| (hr) | (Slow Release) | (Fast Release) | (Fast Release) |
| 0.5 | 18.8 | 21.3 | 20.1 |
| 1 | 27.8 | 32.3 | 31.7 |
| 2 | 40.5 | 47.4 | 46.9 |
| 3 | 50.2 | 58.5 | 57.9 |
| 4 | 58.1 | 66.9 | 66.3 |
| 5 | 64.7 | 73.5 | 74.0 |
| 6 | 70.2 | 78.6 | 83.1 |
| 8 | 79.0 | 86.0 | 92.0 |
| 10 | 85.3 | 90.6 | 95.8 |
| 12 | 89.8 | 93.4 | 97.3 |

### Clinical Studies

Three clinical studies were conducted to assess the bioavailability (rate and extent of absorption) of oxymorphone. Study 1 addressed the relative rates of absorption of controlled release (CR) oxymorphone tablets (of Examples 2 and 3) and oral oxymorphone solution in fasted patients. Study 2 addressed the relative rates of absorption of CR oxymorphone tablets (of Examples 2 and 3) and oral oxymorphone solution in fed patients. Study 3 addressed the relative rates of absorption of CR oxymorphone tablets (of Example 4) and oral oxymorphone solution in fed and fasted patients.

The blood plasma levels set forth herein as appropriate to achieve the objects of the present invention are mean blood plasma levels. As an example, if the blood plasma level of oxymorphone in a patient 12 hours after administration of a tablet is said to be at least 0.5 ng/ml, any particular individual may have lower blood plasma levels after 12 hours. However, the mean minimum concentration should meet the limitation set forth. To determine mean parameters, a study should be performed with a minimum of 8 adult subjects, in a manner acceptable for filing an application for drug approval with the US Food and Drug Administration. In cases where large fluctuations are found among patients, further testing may be necessary to accurately determine mean values.

For all studies, the following procedures were followed, unless otherwise specified for a particular study.

The subjects were not to consume any alcohol-, caffeine-, or xanthine-containing foods or beverages for 24 hours prior to receiving study medication for each study period. Subjects were to be nicotine and tobacco free for at least 6 months prior to enrolling in the study. In addition, over-the-counter medications were prohibited 7 days prior to dosing and during the study. Prescription medications were not allowed 14 days prior to dosing and during the study.

Pharmacokinetic and Statistical Methods

The following pharmacokinetic parameters were computed from the plasma oxymorphone concentration-time data:

AUC₍₀₋ₜ₎ Area under the drug concentration-time curve from time zero to the time of the last quantifiable concentration (Ct), calculated using linear trapezoidal summation.

AUC_{(0-inf)} Area under the drug concentration-time curve from time zero to infinity. AUC_{(0-inf)}=AUC₍₀₋ₜ₎+Ct/Kₑₗ, where Kₑₗ is the terminal elimination rate constant.

AUC₍₀₋₂₄₎ Partial area under the drug concentration-time curve from time zero to 24 hours.

Cₘₐₓ Maximum observed drug concentration.

Tₘₐₓ Time of the observed maximum drug concentration.

Kₑₗ Elimination rate constant based on the linear regression of the terminal linear portion of the LN (concentration) time curve.

Terminal elimination rate constants for use in the above calculations were in turn computed using linear regression of a minimum of three time points, at least two of which were consecutive. Kₑₗ values for which correlation coefficients were less than or equal to 0.8 were not reported in the pharmacokinetic parameter tables or included in the statistical analysis. Thus AUC_{(0-inf)} was also not reported in these cases.

A parametric (normal-theory) general linear model was applied to each of the above parameters (excluding Tₘₐₓ), and the LN-transformed parameters Cₘₐₓ, AUC₍₀₋₂₄₎, AUC₍₀₋ₜ₎, and AUC_{(0-inf)}. Initially, the analysis of variance (ANOVA) model included the following factors: treatment, sequence, subject within sequence, period, and carryover effect. If carryover effect was not significant, it was dropped from the model. The sequence effect was tested using the subject within sequence mean square, and all other main effects were tested using the residual error (error mean square).

Plasma oxymorphone concentrations were listed by subject at each collection time and summarized using descriptive statistics. Pharmacokinetic parameters were also listed by subject and summarized using descriptive statistics.

Study 1—Two Controlled Release Formulations; Fasted Patients

Healthy volunteers received a single oral dose of 20 mg CR oxymorphone taken with 240 ml water after a 10-hour fast. Subjects received the tablets of Example 2 (Treatment 1A) or Example 3 (Treatment 1B). Further subjects were given a single oral dose of 10 mg/10 ml oxymorphone solution in 180 ml apple juice followed with 60 ml water (Treatment 1C). The orally dosed solution was used to simulate an immediate release (IR) dose.

This study had a single-center, open-label, randomized, three-way crossover design using fifteen subjects. Subjects were in a fasted state following a 10-hour overnight fast. There was a 14-day washout interval between the three dose administrations. The subjects were confined to the clinic during each study period. Subjects receiving Treatment 1C were confined for 18 hours and subjects receiving Treatments 1A or 1B were confined for 48 hours after dosing. Ten-milliliter blood samples were collected during each study period at the 0 hour (predose), and at 0.5, 1, 1.5, 2, 3, 4, 5, 6, 7, 8, 10, 12, 14, 16, 18, 20, 24, 28, 32, 36, and 48 hours postdose for subjects receiving Treatment 1A or 1B and 0, 0.25, 0.5, 0.75, 1, 1.25, 1.5, 1.75, 2, 2.5, 3, 4, 5, 6, 7, 8, 10, 12, 14, 16, and 18 hours post-dose. The mean plasma concentration of oxymorphone versus time for each treatment across all subjects is shown in table 5.

TABLE 5
Mean Plasma Concentration vs. Time (ng/ml)

| Time (hr) | Treatment 1A | Treatment 1B | Treatment 1C |
|---|---|---|---|
| 0 | 0.000 | 0.000 | 0.0000 |
| 0.25 | | | 0.9489 |
| 0.5 | 0.2941 | 0.4104 | 1.3016 |
| 0.75 | | | 1.3264 |
| 1 | 0.5016 | 0.7334 | 1.3046 |
| 1.25 | | | 1.2041 |
| 1.5 | 0.5951 | 0.8192 | 1.0813 |
| 1.75 | | | 0.9502 |
| 2 | 0.6328 | 0.7689 | 0.9055 |
| 2.5 | | | 0.7161 |
| 3 | 0.5743 | 0.7341 | 0.6689 |
| 4 | 0.5709 | 0.6647 | 0.4879 |
| 5 | 0.7656 | 0.9089 | 0.4184 |
| 6 | 0.7149 | 0.7782 | 0.3658 |
| 7 | 0.6334 | 0.6748 | 0.3464 |
| 8 | 0.5716 | 0.5890 | 0.2610 |
| 10 | 0.4834 | 0.5144 | 0.2028 |
| 12 | 0.7333 | 0.6801 | 0.2936 |
| 14 | 0.6271 | 0.6089 | 0.2083 |
| 16 | 0.4986 | 0.4567 | 0.1661 |
| 18 | 0.4008 | 0.3674 | 0.1368 |
| 20 | 0.3405 | 0.2970 | |
| 24 | 0.2736 | 0.2270 | |
| 28 | 0.3209 | 0.2805 | |
| 32 | 0.2846 | 0.2272 | |
| 36 | 0.2583 | 0.1903 | |
| 48 | 0.0975 | 0.0792 | |

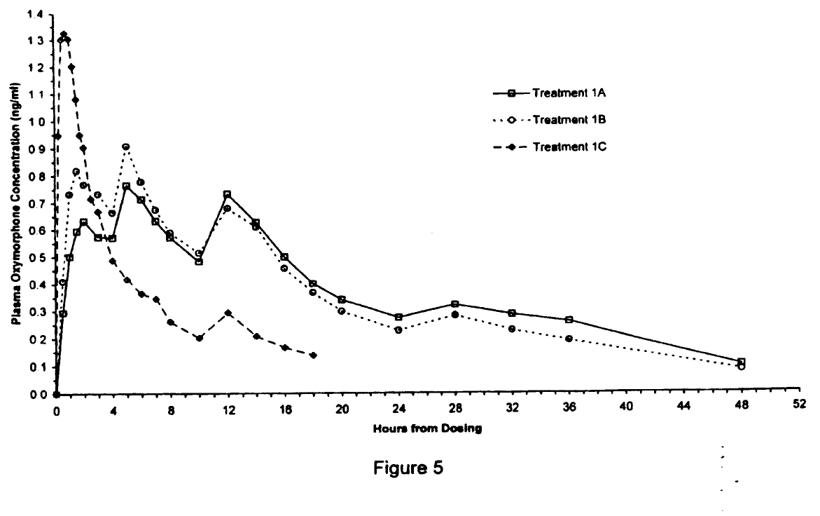

The results are shown graphically in FIG. 5. In both Table 5 and FIG. 5, the results are normalized to a 20 mg dosage. The immediate release liquid of Treatment 1C shows a classical curve, with a high and relatively narrow peak, followed by an exponential drop in plasma concentration. However, the controlled release oxymorphone tablets exhibit triple peaks in blood plasma concentration. The first peak occurs (on average) at around 3 hours. The second peak of the mean blood plasma concentration is higher than the first, occurring around 6-7 hours, on average).

Occasionally, in an individual, the first peak is higher than the second, although generally this is not the case. This makes it difficult to determine the time to maximum blood plasma concentration (Tₘₐₓ) because if the first peak is higher than the second, maximum blood plasma concentration (Cₘₐₓ) occurs much earlier (at around 3 hours) than in the usual case where the second peak is highest. Therefore, when we refer to the time to peak plasma concentration (Tₘₐₓ) unless otherwise specified, we refer to the time to the second peak. Further, when reference is made to the second peak, we refer to the time or blood plasma concentration at the point where the blood plasma concentration begins to drop the second time. Generally, where the first peak is higher than the second, the difference in the maximum blood plasma concentration at the two peaks is small. Therefore, this difference (if any) was ignored and the reported Cₘₐₓ was the true maximum blood plasma concentration and not the concentration at the second peak.

TABLE 6
Pharmacokinetic Parameters of Plasma Oxymorphone for Study 1

| | | |
|---|---|---|
| Treatment 1A | Treatment 1B | Treatment 1C |

| | Mean | SD | Mean | SD | Mean | SD |
|---|---|---|---|---|---|---|
| Cₘₐₓ | 0.8956 | 0.2983 | 1.0362 | 0.3080 | 2.9622 | 1.0999 |
| Tₘₐₓ | 7.03 | 4.10 | 4.89 | 3.44 | 0.928 | 0.398 |
| AUC₍₀₋ₜ₎ | 17.87 | 6.140 | 17.16 | 6.395 | 14.24 | 5.003 |
| AUC_{(0-inf)} | 19.87 | 6.382 | 18.96 | 6.908 | 16.99 | 5.830 |
| T_{1/2el} | 10.9 | 2.68 | 11.4 | 2.88 | 6.96 | 4.61 |

Units:
Cₘₐₓ in ng/ml,
Tₘₐₓ in hours,
AUC in ng * hr/ml,
T_{1/2el} in hours.

Relative bioavailability determinations are set forth in Tables 7 and 8. For these calculations, AUC was normalized for all treatments to a 20 mg dose.

TABLE 7
Relative Bioavailability (Fᵣₑₗ) Determination Based on AUC_{(0-inf)}

| Fᵣₑₗ (1A vs. 1C) | Fᵣₑₗ (1B vs. 1C) | Fᵣₑₗ (1A vs. 1B) |
|---|---|---|
| 1.193 .±. 0.203 | 1.121 .±. 0.211 | 1.108 .±. 0.152 |

TABLE 8
Relative Bioavailability Determination Based on AUC₍₀₋₁₈₎

| Fᵣₑₗ (1A vs. 1C) | Fᵣₑₗ (1B vs. 1C) | Fᵣₑₗ (1A vs. 1B) |
|---|---|---|
| 0.733 .±. 0.098 | 0.783 .±. 0.117 | 0.944 .±. 0.110 |

Study 2—Two CR Formulations; Fed Patients

Healthy volunteers received a single oral dose of 20 mg CR oxymorphone taken with 240 ml water in a fed state. Subjects received the tablets of Example 2 (Treatment 2A) or Example 3 (Treatment 2B). Further subjects were given a single oral dose of 10 mg/10 ml oxymorphone solution in 180 ml apple juice followed with 60 ml water (Treatment 2C). The orally dosed solution was used to simulate an immediate release (IR) dose.

This study had a single-center, open-label, randomized, three-way crossover design using fifteen subjects. The subjects were in a fed state, after a 10-hour overnight fast followed by a standardized FDA high-fat breakfast. There was a 14-day washout interval between the three dose administrations. The subjects were confined to the clinic during each study period. Subjects receiving Treatment 2C were confined for 18 hours and subjects receiving Treatments 2A or 2B were confined for 48 hours after dosing. Ten-milliliter blood samples were collected during each study period at the 0 hour (predose), and at 0.5, 1, 1.5, 2, 3, 4, 5, 6, 7, 8, 10, 12, 14, 16, 18, 20, 24, 28, 32, 36, and 48 hours postdose for subjects receiving Treatment 2A or 2B and 0, 0.25, 0.5, 0.75, 1, 1.25, 1.5, 1.75, 2, 2.5, 3, 4, 5, 6, 7, 8, 10, 12, 14, 16, and 18 hours postdose. The mean plasma concentration of oxymorphone versus time for each treatment across all subjects is shown in table 9.

TABLE 9
Mean Plasma Concentration vs. Time (ng/ml)

| Time (hr) | Treatment 2A | Treatment 2B | Treatment 2C |
|---|---|---|---|
| 0 | 0.000 | 0.000 | 0.0000 |
| 0.25 | | | 1.263 |
| 0.5 | 0.396 | .0553 | 1.556 |
| 0.75 | | | 1.972 |
| 1 | 0.800 | 1.063 | 1.796 |
| 1.25 | | | 1.795 |
| 1.5 | 1.038 | 1.319 | 1.637 |
| 1.75 | | | 1.467 |
| 2 | 1.269 | 1.414 | 1.454 |
| 2.5 | | | 1.331 |
| 3 | 1.328 | 1.540 | 1.320 |
| 4 | 1.132 | 1.378 | 1.011 |
| 5 | 1.291 | 1.609 | 0.731 |
| 6 | 1.033 | 1.242 | 0.518 |
| 7 | 0.941 | 0.955 | 0.442 |
| 8 | 0.936 | 0.817 | 0.372 |
| 10 | 0.669 | 0.555 | 0.323 |
| 12 | 0.766 | 0.592 | 0.398 |
| 14 | 0.641 | 0.519 | 0.284 |
| 16 | 0.547 | 0.407 | 0.223 |
| 18 | 0.453 | 0.320 | 0.173 |
| 20 | 0.382 | 0.280 | |
| 24 | 0.315 | 0.254 | |
| 28 | 0.352 | 0.319 | |
| 32 | 0.304 | 0.237 | |
| 36 | 0.252 | 0.207 | |
| 48 | 0.104 | 0.077 | |

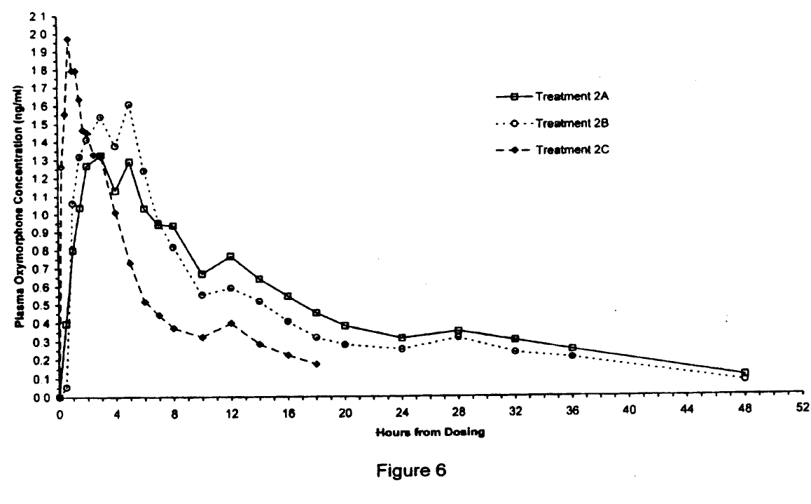

The results are shown graphically in FIG. 6. Again, the results have been normalized to a 20 mg dosage. As with Study 1, the immediate release liquid of Treatment 2C shows a classical curve, with a high and relatively narrow peak, followed by an exponential drop in plasma concentration, while the controlled release oxymorphone tablets exhibit triple peaks in blood plasma concentration. Thus, again when we refer to the time to peak plasma concentration (Tₘₐₓ) unless otherwise specified, we refer to the time to the second peak.

TABLE 10
Pharmacokinetic Parameters of Plasma Oxymorphone for Study 2

| | Treatment | | Treatment | | | |
|---|---|---|---|---|---|---|
| | 2A | | 2B | | Treatment 2C | |
| | Mean | SD | Mean | SD | Mean | SD |
| Cₘₐₓ | 1.644 | 0.365 | 1.944 | 0.465 | 4.134 | 0.897 |
| Tₘₐₓ | 3.07 | 1.58 | 2.93 | 1.64 | 0.947 | 0.313 |
| AUC₍₀₋ₜ₎ | 22.89 | 5.486 | 21.34 | 5.528 | 21.93 | 5.044 |
| AUC_{(0-inf)} | 25.28 | 5.736 | 23.62 | 5.202 | 24.73 | 6.616 |
| T_{1/2el} | 12.8 | 3.87 | 11.0 | 3.51 | 5.01 | 2.02 |

Units:
Cₘₐₓ in ng/ml,
Tₘₐₓ in hours,
AUC in ng * hr/ml,
T_{1/2el} in hours.

In Table 10, the Tₘₐₓ has a large standard deviation due to the two comparable peaks in blood plasma concentration. Relative bioavailability determinations are set forth in Tables 11 and 12.

TABLE 11
Relative Bioavailability Determination Based on AUC_{(0-inf)}

| Fᵣₑₗ (2A vs. 2C) | Fᵣₑₗ (2B vs. 2C) | Fᵣₑₗ (2A vs. 2B) |
|---|---|---|
| 1.052 .±. 0.187 | 0.949 .±. 0.154 | 1.148 .±. 0.250 |

TABLE 12
Relative bioavailability Determination Based on AUC₍₀₋₁₈₎

| Fᵣₑₗ (2A vs. 2C) | Fᵣₑₗ (2B vs. 2C) | Fᵣₑₗ (2A vs. 2B) |
|---|---|---|
| 0.690 .±. 0.105 | 0.694 .±. 0.124 | 1.012 .±. 0.175 |

As may be seen from tables 5 and 10 and FIGS. 1 and 2, the Cₘₐₓ for the CR tablets (treatments 1A, 1B, 2A and 2B) is considerably lower, and the T max much higher than for the immediate release oxymorphone. The blood plasma level of oxymorphone remains high well past the 8 (or even the 12) hour dosing interval desired for an effective controlled release tablet.

Study 3—One Controlled Release Formulation; Fed and Fasted Patients

This study had a single-center, open-label, analytically blinded, randomized, four-way crossover design. Subjects randomized to Treatment 3A and Treatment 3C, as described below, were in a fasted state following a 10-hour overnight fast. Subjects randomized to Treatment 3B and Treatment 3D, as described below, were in the fed state, having had a high fat meal, completed ten minutes prior to dosing. There was a 14-day washout interval between the four dose administrations. The subjects were confined to the clinic during each study period. Subjects assigned to receive Treatment 3A and Treatment 3B were discharged from the clinic on Day 3 following the 48-hour procedures, and subjects assigned to receive Treatment 3C and Treatment 3D were discharged from the clinic on Day 2 following the 36-hour procedures. On Day 1 of each study period the subjects received one of four treatments:

Treatments 3A and 3B: Oxymorphone controlled release 20 mg tablets from Example 3. Subjects randomized to Treatment 3A received a single oral dose of one 20 mg oxymorphone controlled release tablet taken with 240 ml of water after a 10-hour fasting period. Subjects randomized to Treatment 3B received a single oral dose of one 20 mg oxymorphone controlled release tablet taken with 240 ml of water 10 minutes after a standardized high fat meal.

Treatments 3C and 3D: oxymorphone HCl solution, USP, 1.5 mg/ml 10 ml vials. Subjects randomized to Treatment 3C received a single oral dose of 10 mg (6.7 ml) oxymorphone solution taken with 240 ml of water after a 10-hour fasting period. Subjects randomized to Treatment 3D received a single oral dose of 10 mg (6.7 ml) oxymorphone solution taken with 240 ml of water 10 minutes after a standardized high-fat meal.

A total of 28 male subjects were enrolled in the study, and 24 subjects completed the study. The mean age of the subjects was 27 years (range of 19 through 38 years), the mean height of the subjects was 69.6 inches (range of 64.0 through 75.0 inches), and the mean weight of the subjects was 169.0 pounds (range 117.0 through 202.0 pounds).

A total of 28 subjects received at least one treatment. Only subjects who completed all 4 treatments were included in the summary statistics and statistical analysis.

Blood samples (7 ml) were collected during each study period at the 0 hour (predose), and at 0.5, 1, 1.5, 2, 3, 4, 5, 6, 8, 10, 12, 14, 16, 20, 24, 30, 36, and 48 hours post-dose (19 samples) for subjects randomized to Treatment 3A and Treatment 3B. Blood samples (7 ml) were collected during each study period at the 0 hour (predose), and at 0.25, 0.5, 0.75, 1, 1.25, 1.5, 1.75, 2, 3, 4, 5, 6, 8, 10, 12, 14, 16, 20, and 36 hours post-dose (21 samples) for subjects randomized to Treatment 3C and Treatment 3D.

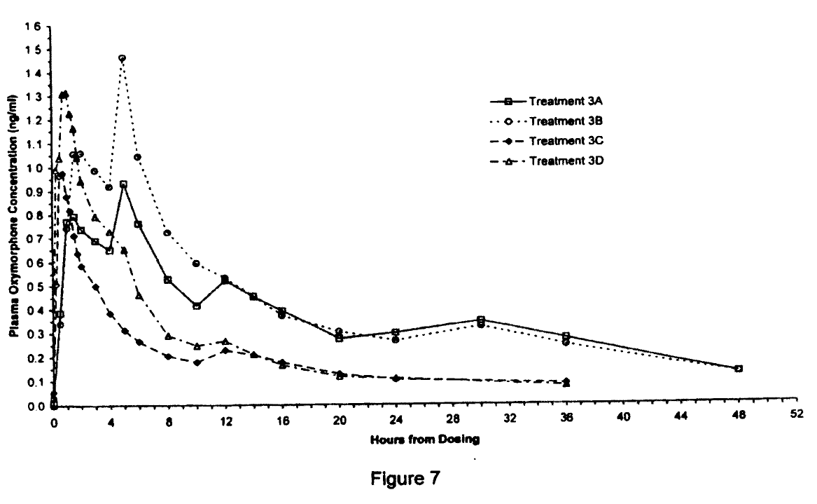

The mean oxymorphone plasma concentration versus time curves for Treatments 3A, 3B, 3C, and 3D are presented in FIG. 7. The results have been normalized to a 20 mg dosage. The data is contained in Table 13. The arithmetic means of the plasma oxymorphone pharmacokinetic parameters and the statistics for all Treatments are summarized in Table 1.

TABLE 13
Mean Plasma Concentration vs. Time (ng/ml)

| | Treatment | Treatment | Treatment | Treatment |
|---|---|---|---|---|
| Time (hr) | 3A | 3B | 3C | 3D |
| 0 | 0.0084 | 0.0309 | 0.0558 | 0.0000 |
| 0.25 | | | 0.5074 | 0.9905 |
| 0.5 | 0.3853 | 0.3380 | 0.9634 | 1.0392 |
| 0.75 | | | 0.9753 | 1.3089 |
| 1 | 0.7710 | 0.7428 | 0.8777 | 1.3150 |
| 1.25 | | | 0.8171 | 1.2274 |
| 1.5 | 0.7931 | 1.0558 | 0.7109 | 1.1638 |
| 1.75 | | | 0.6357 | 1.0428 |
| 2 | 0.7370 | 1.0591 | 0.5851 | 0.9424 |
| 3 | 0.6879 | 0.9858 | 0.4991 | 0.7924 |
| 4 | 0.6491 | 0.9171 | 0.3830 | 0.7277 |
| 5 | 0.9312 | 1.4633 | 0.3111 | 0.6512 |
| 6 | 0.7613 | 1.0441 | 0.2650 | 0.4625 |
| 8 | 0.5259 | 0.7228 | 0.2038 | 0.2895 |
| 10 | 0.4161 | 0.5934 | 0.1768 | 0.2470 |
| 12 | 0.5212 | 0.5320 | 0.2275 | 0.2660 |
| 14 | 0.4527 | 0.4562 | 0.2081 | 0.2093 |
| 16 | 0.3924 | 0.3712 | 0.1747 | 0.1623 |
| 20 | 0.2736 | 0.3021 | 0.1246 | 0.1144 |
| 24 | 0.2966 | 0.2636 | 0.1022 | 0.1065 |
| 30 | 0.3460 | 0.3231 | | |
| 36 | 0.2728 | 0.2456 | 0.0841 | 0.0743 |
| 48 | 0.1263 | 0.1241 | | |

TABLE 14
Pharmacokinetic Parameters of Plasma Oxymorphone for Study 3

| | Treatment 3A | | Treatment 3B | | Treatment 3C | | Treatment 3D | |
|---|---|---|---|---|---|---|---|---|
| | Mean | SD | Mean | SD | Mean | SD | Mean | SD |
| Cₘₐₓ | 1.1410 | 0.4537 | 1.7895 | 0.6531 | 2.2635 | 1.0008 | 3.2733 | 1.3169 |
| Tₘₐₓ | 5.57 | 7.14 | 5.65 | 9.39 | 0.978 | 1.14 | 1.11 | 0.768 |
| AUC₍₀₋ₜ₎ | 11.64 | 3.869 | 14.27 | 4.976 | 12.39 | 4.116 | 17.30 | 5.259 |
| AUC_{(0-inf)} | 17.71 | 8.471 | 19.89 | 6.408 | 14.53 | 4.909 | 19.28 | 6.030 |
| T_{1/2el} | 19.29 | 5.028 | 21.29 | 6.559 | 18.70 | 6.618 | 25.86 | 10.03 |
| | 12.3 | 3.99 | 12.0 | 3.64 | 16.2 | 11.4 | 20.6 | 19.3 |

The relative bioavailability calculations are summarized in tables 15 and 16.

TABLE 15
Relative Bioavailability Determination Based on AUC_{(0-inf)}

| Fᵣₑₗ (3A vs. 3C) | Fᵣₑₗ (3B vs. 3D) | Fᵣₑₗ (3D vs. 3C) | Fᵣₑₗ (3B vs. 3A) |
|---|---|---|---|
| 1.040 .±. | 0.8863 .±. 0.2569 | 1.368 .±. 0.4328 | 1.169 .±. 0.2041 |
| 0.1874 | | | |

TABLE 16
Relative bioavailability Determination Based on AUC₍₀₋₂₄₎

| Fᵣₑₗ (3A vs. 3C) | Fᵣₑₗ (3B vs. 3D) | Fᵣₑₗ (3D vs. 3C) | Fᵣₑₗ (3B vs. 3A) |
|---|---|---|---|
| 0.9598 .±. | 0.8344 .±. 0.100 | 1.470 .±. 0.3922 | 1.299 .±. 0.4638 |
| 0.2151 | | | |

The objectives of this study were to assess the relative bioavailability of oxymorphone from oxymorphone controlled release (20 mg) compared to oxymorphone oral solution (10 mg) under both fasted and fed conditions, and to determine the effect of food on the bioavailability of oxymorphone from the controlled release formulation, oxymorphone CR, and from the oral solution.

The presence of a high fat meal had a substantial effect on the oxymorphone Cₘₐₓ, but less of an effect on oxymorphone AUC from oxymorphone controlled release tablets. Least Squares (LS) mean Cₘₐₓ was 58% higher and LS mean AUC₍₀₋ₜ₎ and AUC_{(0-inf)} were 18% higher for the fed condition (Treatment B) compared to the fasted condition (Treatment A) based on LN-transformed data. This was consistent with the relative bioavailability determination from AUC_{(0-inf)} since mean Fᵣₑₗ was 1.17. Mean Tₘₐₓ values were similar (approximately 5.6 hours), and no significant difference in Tₘₐₓ was shown using nonparametric analysis. Half value durations were significantly different between the two treatments.

The effect of food on oxymorphone bioavailability from the oral solution was more pronounced, particularly in terms of AUC. LS mean Cₘₐₓ was 50% higher and LS mean AUC₍₀₋ₜ₎ and AUC_{(0-inf)} were 32-34% higher for the fed condition (Treatment D) compared to the fasted condition (Treatment C) based on LN-transformed data. This was consistent with the relative bioavailability determination from AUC_{(0-inf)} since mean Fᵣₑₗ was 1.37. Mean Tₘₐₓ (approximately 1 hour) was similar for the two treatments and no significant difference was shown.

Under fasted conditions, oxymorphone controlled release 20 mg tablets exhibited similar extent of oxymorphone availability compared to 10 mg oxymorphone oral solution normalized to a 20 mg dose (Treatment A versus Treatment C). From LN-transformed data, LS mean AUC₍₀₋ₜ₎ was 17% higher for oxymorphone CR, whereas LS mean AUC_{(0-inf)} values were nearly equal (mean ratio=99%). Mean Fᵣₑₗ values calculated from AUC_{(0-inf)} and AUC₍₀₋₂₄₎, (1.0 and 0.96, respectively) also showed similar extent of oxymorphone availability between the two treatments.

As expected, there were differences in parameters reflecting rate of absorption. LS mean Cₘₐₓ was 49% lower for oxymorphone controlled release tablets compared to the dose-normalized oral solution, based on LN-transformed data. Half-value duration was significantly longer for the controlled release formulation (means, 12 hours versus 2.5 hours).

Under fed conditions, oxymorphone availability from oxymorphone controlled release 20 mg was similar compared to 10 mg oxymorphone oral solution normalized to a 20 mg dose (Treatment B versus Treatment D). From LN-transformed data, LS mean AUC_{(0-inf)} was 12% lower for oxymorphone CR. Mean Fᵣₑₗ values calculated from AUC_{(0-inf)} and AUC₍₀₋₂₄₎, (0.89 and 0.83 respectively) also showed similar extent of oxymorphone availability from the tablet. As expected, there were differences in parameters reflecting rate of absorption. LS mean Cₘₐₓ was 46% lower for oxymorphone controlled release tablets compared to the dose-normalized oral solution, based on LN-transformed data. Mean Tₘₐₓ was 5.7 hours for the tablet compared to 1.1 hours for the oral solution. Half-value duration was significantly longer for the controlled release formulation (means, 7.8 hours versus 3.1 hours).

The presence of a high fat meal did not appear to substantially affect the availability of 6-hydroxyoxymorphone following administration of oxymorphone controlled release tablets. LS mean ratios were 97% for AUC₍₀₋ₜ₎ and 91% for Cₘₐₓ(Treatment B versus A), based on LN-transformed data. This was consistent with the relative bioavailability determination from AUC₍₀₋₂₄₎, since mean Fᵣₑₗ was 0.97. Mean Tₘₐₓ was later for the fed treatment compared to the fasted treatment (5.2 and 3.6 hours, respectively), and difference was significant.

Under fasted conditions, oxymorphone controlled release 20 mg tablets exhibited similar availability of 6-hydroxyoxymorphone compared to 10 mg oxymorphone oral solution normalized to a 20 mg dose (Treatment A versus Treatment C). From LN-transformed data, LS mean ratio for AUC₍₀₋ₜ₎ was 104.5%. Mean Fᵣₑₗ (0.83) calculated from AUC₍₀₋₂₄₎ also showed similar extent of oxymorphone availability between the two treatments. Mean Tₘₐₓ was 3.6 hours for the tablet compared to 0.88 for the oral solution. Half-value duration was significantly longer for the controlled release formulation (means, 11 hours versus 2.2 hours).

Under fed conditions, availability of 6-hydroxyoxymorphone from oxymorphone controlled release 20 mg was similar compared to 10 mg oxymorphone oral solution normalized to a 20 mg dose (Treatment B versus Treatment D). From LN-transformed data, LS mean AUC₍₀₋ₜ₎ was 14% higher for oxymorphone CR. Mean Fᵣₑₗ (0.87) calculated from AUC₍₀₋₂₄₎ also indicated similar extent of availability between the treatments. Mean Tₘₐₓ was 5.2 hours for the tablet compared to 1.3 hour for the oral solution. Half-value duration was significantly longer for the controlled release formulation (means, 14 hours versus 3.9 hours).

The extent of oxymorphone availability from oxymorphone controlled release 20 mg tablets was similar under fed and fasted conditions since there was less than a 20% difference in LS mean AUC₍₀₋ₜ₎ and AUC_{(0-inf)} values for each treatment, based on LN-transformed data. Tₘₐₓ was unaffected by food; however, LS mean Cₘₐₓ was increased 58% in the presence of the high fat meal. Both rate and extent of oxymorphone absorption from the oxymorphone oral solution were affected by food since LS mean Cₘₐₓ and AUC values were increased approximately 50 and 30%, respectively. Tₘₐₓ was unaffected by food. Under both fed and fasted conditions, oxymorphone controlled release tablets exhibited similar extent of oxymorphone availability compared to oxymorphone oral solution since there was less than a 20% difference in LS mean AUC(0-t) and AUC(0-inf) values for each treatment.

Bioavailability of 6-hydroxyoxymorphone following oxymorphone controlled release 20 mg tablets was also similar under fed and fasted conditions since there was less than a 20% difference in LS mean Cₘₐₓ and AUC values for each treatment. Tₘₐₓ was later for the fed condition. The presence of food did not affect the extent of availability from oxymorphone oral solution since LS mean AUC values were less than 20% different. However, Cₘₐₓ was decreased 35% in the presence of food. Tₘₐₓ was unaffected by food. Under both fed and fasted conditions, oxymorphone controlled release tablets exhibited similar extent of availability compared to oxymorphone oral solution since there was less than a 20% difference in LS mean AUC values for each treatment.

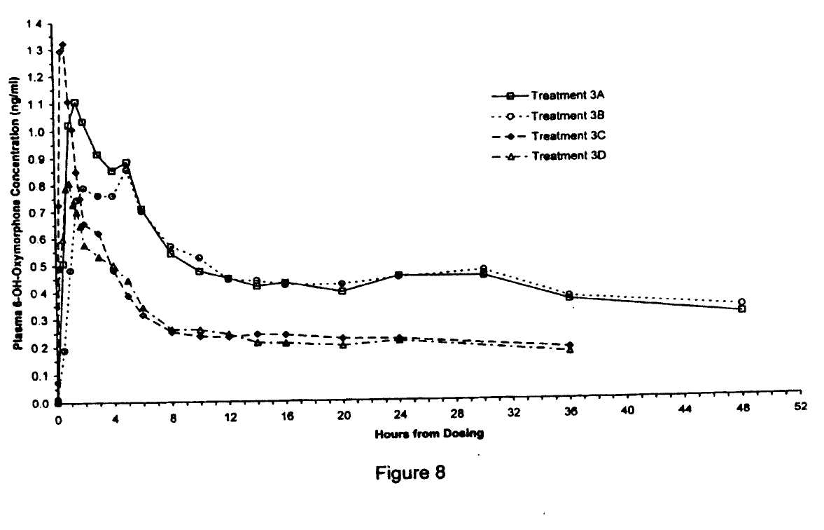

The mean 6-OH oxymorphone plasma concentration versus time curves for Treatments 3A, 3B, 3C, and 3D are presented in FIG. 8. The data is contained in Table 17.

TABLE 17
Mean Plasma Concentration vs. Time (ng/ml)
6-Hydroxyoxymorphone

| | Treatment | Treatment | Treatment | Treatment |
|---|---|---|---|---|
| Time (hr) | 3A | 3B | 3C | 3D |
| 0 | 0.0069 | 0.0125 | 0.0741 | 0.0000 |
| 0.25 | | | 0.7258 | 0.4918 |
| 0.5 | 0.5080 | 0.1879 | 1.2933 | 0.5972 |
| 0.75 | | | 1.3217 | 0.7877 |
| 1 | 1.0233 | 0.4830 | 1.1072 | 0.8080 |
| 1.25 | | | 1.0069 | 0.7266 |
| 1.5 | 1.1062 | 0.7456 | 0.8494 | 0.7001 |
| 1.75 | | | 0.7511 | 0.6472 |
| 2 | 1.0351 | 0.7898 | 0.6554 | 0.5758 |
| 3 | 0.9143 | 0.7619 | 0.6196 | 0.5319 |
| 4 | 0.8522 | 0.7607 | 0.4822 | 0.5013 |
| 5 | 0.8848 | 0.8548 | 0.3875 | 0.4448 |
| 6 | 0.7101 | 0.7006 | 0.3160 | 0.3451 |
| 8 | 0.5421 | 0.5681 | 0.2525 | 0.2616 |
| 10 | 0.4770 | 0.5262 | 0.2361 | 0.2600 |
| 12 | 0.4509 | 0.4454 | 0.2329 | 0.2431 |
| 14 | 0.4190 | 0.4399 | 0.2411 | 0.2113 |
| 16 | 0.4321 | 0.4230 | 0.2385 | 0.2086 |
| 20 | 0.3956 | 0.4240 | 0.2234 | 0.1984 |
| 24 | 0.4526 | 0.4482 | 0.2210 | 0.2135 |
| 30 | 0.4499 | 0.4708 | | |
| 36 | 0.3587 | 0.3697 | 0.1834 | 0.1672 |
| 48 | 0.3023 | 0.3279 | | |

TABLE 18
Pharmacokinetic Parameters of Plasma 6-Hydroxyoxymorphone for Study 3

| | Treatment 3A | | Treatment 3B | | Treatment 3C | | Treatment 3D | |
|---|---|---|---|---|---|---|---|---|
| | Mean | SD | Mean | SD | Mean | SD | Mean | SD |
| Cₘₐₓ | 1.2687 | 0.5792 | 1.1559 | 0.4848 | 1.5139 | 0.7616 | 0.9748 | 0.5160 |
| Tₘₐₓ | 3.61 | 7.17 | 5.20 | 9.52 | 0.880 | 0.738 | 1.30 | 1.04 |
| AUC₍ₒ₋ₜ₎ | 22.47 | 10.16 | 22.01 | 10.77 | 10.52 | 4.117 | 9.550 | 4.281 |
| AUC_{(o-inf)} | 38.39 | 23.02 | 42.37 | 31.57 | 20.50 | 7.988 | 23.84 | 11.37 |
| T_{1/2el} | 39.1 | 36.9 | 39.8 | 32.6 | 29.3 | 12.0 | 44.0 | 35.00 |

Study 4—Controlled Release 20 mg Vs Immediate Release 10 mg

A study was conducted to compare the bioavailability and pharmacokinetics of controlled release and immediate release oxymorphone tablets under single-dose and multiple-dose (steady state) conditions. For the controlled release study, healthy volunteers received a single dose of a 20 mg controlled release oxymorphone table on the morning of Day 1. Beginning on the morning of Day 3, the volunteers were administered a 20 mg controlled release oxymorphone tablet every 12 hours through the morning dose of Day 9. For the immediate release study, healthy volunteers received a single 10 mg dose of an immediate release oxymorphone tablet on the morning of Day 1. On the morning of Day 3, additional 10 mg immediate release tablets were administered every six hours through the first two doses on Day 9.

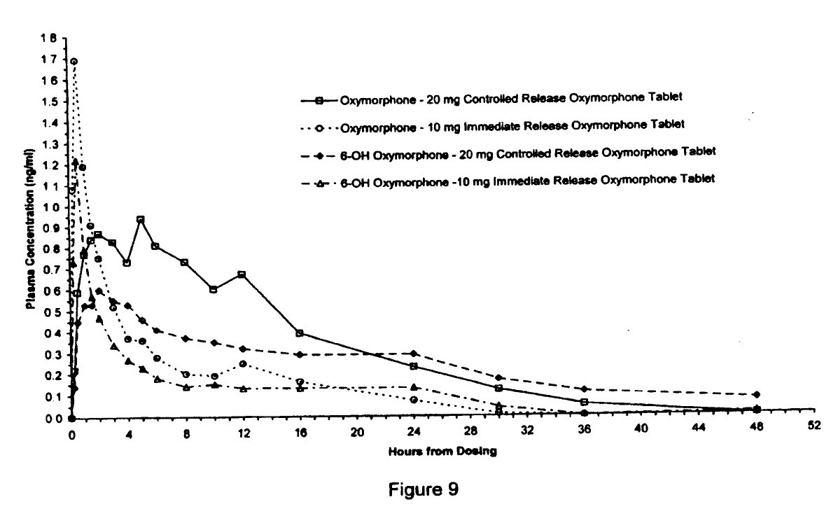

FIG. 9 shows the average plasma concentrations of oxymorphone and 6-hydroxyoxymorphone for all subjects after a single dose either controlled release (CR) 20 mg or immediate release (IR) 10 mg oxymorphone. The data in the figure (as with the other relative experimental data herein) is normalized to a 20 mg dose. The immediate release tablet shows a classical curve, with a high, relatively narrow peak followed by an exponential drop in plasma concentration. The controlled release oxymorphone tablets show a lower peak with extended moderate levels of oxymorphone and 6-hydroxy oxymorphone. Table 19 shows the levels of oxymorphone and 6-hydroxy oxymorphone from FIG. 9 in tabular form.

TABLE 19
Mean Plasma Concentration (ng/ml)

| | |
|---|---|
| Oxymorphone | 6-Hydroxyoxymorphone |

| | Controlled | Immediate | Controlled | Immediate |
|---|---|---|---|---|
| | Release | Release | Release | Release |
| Hour | 20 mg | 10 mg | 20 mg | 10 mg |
| 0.00 | 0.00 | 0.00 | 0.00 | 0.00 |
| 0.25 | 0.22 | 1.08 | 0.14 | 0.73 |
| 0.50 | 0.59 | 1.69 | 0.45 | 1.22 |
| 1.00 | 0.77 | 1.19 | 0.53 | 0.79 |
| 1.50 | 0.84 | 0.91 | 0.53 | 0.57 |
| 2.00 | 0.87 | 0.75 | 0.60 | 0.47 |
| 3.00 | 0.83 | 0.52 | 0.55 | 0.34 |
| 4.00 | 0.73 | 0.37 | 0.53 | 0.27 |
| 5.00 | 0.94 | 0.36 | 0.46 | 0.23 |
| 6.00 | 0.81 | 0.28 | 0.41 | 0.18 |
| 8.00 | 0.73 | 0.20 | 0.37 | 0.14 |
| 10.0 | 0.60 | 0.19 | 0.35 | 0.15 |
| 12.0 | 0.67 | 0.25 | 0.32 | 0.13 |
| 16.0 | 0.39 | 0.16 | 0.29 | 0.13 |
| 24.0 | 0.23 | 0.07 | 0.29 | 0.13 |
| 30.0 | 0.12 | 0.01 | 0.17 | 0.04 |
| 36.0 | 0.05 | 0.00 | 0.11 | 0.00 |
| 48.0 | 0.00 | 0.00 | 0.07 | 0.01 |

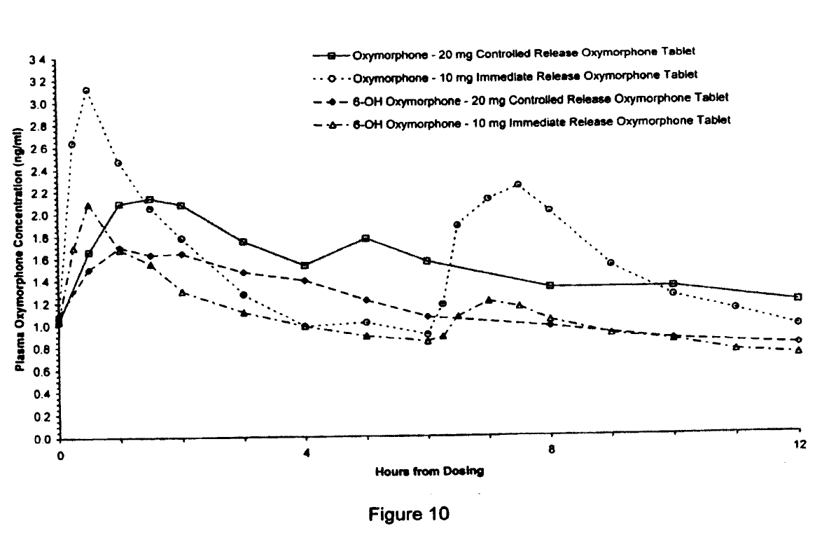

FIG. 10 shows the average plasma concentrations of oxymorphone and 6-hydroxyoxymorphone for all subjects in the steady state test, for doses of controlled release 20 mg tablets and immediate release 10 mg tablets of oxymorphone. The figure shows the plasma concentrations after the final controlled release tablet is given on Day 9, and the final immediate release tablet is given 12 hours thereafter. The steady state administration of the controlled release tablets clearly shows a steady moderate level of oxymorphone ranging from just over 1 ng/ml to almost 1.75 ng/ml over the course of a twelve hour period, where the immediate release tablet shows wide variations in blood plasma concentration. Table 20 shows the levels of oxymorphone and 6-hydroxyoxymorphone from FIG. 10 in tabular form.

TABLE 20
Summary of Mean Plasma Concentration (ng/ml)

| | |
|---|---|
| Oxymorphone | 6-Hydroxyoxymorphone |

| | | Controlled | Immediate | Controlled | Immediate |
|---|---|---|---|---|---|
| | | Release | Release | Release | Release |
| Day | Hour | 20 mg | 10 mg | 20 mg | 10 mg |
| 4 | 0.00 | 1.10 | 0.75 | 0.89 | 0.72 |
| 5 | 0.00 | 1.12 | 0.84 | 1.15 | 0.88 |
| 6 | 0.00 | 1.20 | 0.92 | 1.15 | 0.87 |
| 7 | 0.00 | 1.19 | 0.91 | 1.27 | 1.00 |
| 8 | 0.00 | 1.19 | 0.86 | 1.29 | 0.98 |
| 9 | 0.00 | 1.03 | 1.07 | 1.09 | 1.05 |
| | 0.25 | | 2.64 | | 1.70 |
| | 0.50 | | 3.12 | 1.50 | 2.09 |
| | 1.00 | | 2.47 | 1.70 | 1.68 |
| | 1.50 | | 2.05 | 1.63 | 1.55 |
| | 2.00 | | 1.78 | 1.64 | 1.30 |
| | 3.00 | | 1.27 | 1.47 | 1.11 |
| | 4.00 | | 0.98 | 1.39 | 0.98 |
| | 5.00 | | 1.01 | 1.21 | 0.89 |
| | 6.00 | | 0.90 | 1.06 | 0.84 |
| | 6.25 | | 1.17 | | 0.88 |
| | 6.50 | | 1.88 | | 1.06 |
| | 7.00 | | 2.12 | | 1.20 |
| | 7.50 | | 2.24 | | 1.15 |
| | 8.00 | 1.32 | 2.01 | 0.97 | 1.03 |
| | 9.00 | | 1.52 | | 0.90 |
| | 10.0 | 1.32 | 1.24 | 0.85 | 0.84 |
| | 11.0 | | 1.11 | | 0.74 |
| | 12.0 | 1.18 | 0.96 | 0.79 | 0.70 |

TABLE 21
Mean Single-Dose Pharmacokinetic Results

| | Controlled | | Immediate | |
|---|---|---|---|---|
| | Release 20 mg | | Release 10 mg | |
| | | 6-OH- | | 6-OH- |
| | oxymor- | oxymor- | oxymor- | oxymor- |
| | phone | phone | phone | phone |
| AUC₍ₒ₋ₜ₎ | 14.74 | 11.54 | 7.10 | 5.66 |
| AUC_{(o-inf)} | 15.33 | 16.40 | 7.73 | 8.45 |
| Cₘₐₓ(ng/ml) | 1.12 | 0.68 | 1.98 | 1.40 |
| Tₘₐₓ(hr) | 5.00 | 2.00 | 0.50 | 0.50 |
| T½(hr) | 9.25 | 26.09 | 10.29 | 29.48 |

Parent 6-OH oxymorphone AUC₍ₒ₋ₜ₎ values were lower than the parent compound after administration of either dosage form, but the AUC_{(o-inf)} values are slightly higher due to the longer half-life for the metabolite. This relationship was similar for both the immediate-release (IR) and controlled release (CR) dosage forms. As represented by the average plasma concentration graph, the CR dosage form has a significantly longer time to peak oxymorphone concentration and a lower peak oxymorphone concentration. The 6-OH oxymorphone peak occurred sooner than the parent peak following the CR dosage form, and simultaneously with the parent peak following the IR dosage form.

It is important to note that while the present invention is described and exemplified using 20 mg tablets, the invention may also be used with other strengths of tablets. In each strength, it is important to note how a 20 mg tablet of the same composition (except for the change in strength) would act. The blood plasma levels and pain intensity information are provided for 20 mg tablets, however the present invention is also intended to encompass 5 to 80 mg controlled release tablets. For this reason, the blood plasma level of oxymorphone or 6-hydroxyoxymorphone in nanograms per milliliter of blood, per mg oxymorphone (ng/mg-ml) administered is measured. Thus at 0.02 ng/mg-ml, a 5 mg tablet should produce a minimum blood plasma concentration of 0.1 ng/ml. A stronger tablet will produce a higher blood plasma concentration of active molecule, generally proportionally. Upon administration of a higher dose tablet, for example 80 mg, the blood plasma level of oxymorphone and 6-OH oxymorphone may more than quadruple compared to a 20 mg dose, although conventional treatment of low bioavailability substances would lead away from this conclusion. If this is the case, it may be because the body can only process a limited amount oxymorphone at one time. Once the bolus is processed, the blood level of oxymorphone returns to a proportional level.

It is the knowledge that controlled release oxymorphone tablets are possible to produce and effective to use, which is most important, made possible with the high bioavailability of oxymorphone in a controlled release tablet. This also holds true for continuous periodic administration of controlled release formulations. The intent of a controlled release opioid formulation is the long-term management of pain. Therefore, the performance of a composition when administered periodically (one to three times per day) over several days is important. In such a regime, the patient reaches a “steady state” where continued administration will produce the same results, when measured by duration of pain relief and blood plasma levels of pharmaceutical. Such a test is referred to as a “steady state” test and may require periodic administration over an extended time period ranging from several days to a week or more. Of course, since a patient reaches steady state in such a test, continuing the test for a longer time period should not affect the results. Further, when testing blood plasma levels in such a test, if the time period for testing exceeds the interval between doses, it is important the regimen be stopped after the test is begun so that observations of change in blood level and pain relief may be made without a further dose affecting these parameters.

Study 5—Controlled Release 40 mg Vs Immediate Release 4.Times.10 mg Under Fed and Fasting Conditions

The objectives of this study were to assess the relative bioavailability of oxymorphone from oxymorphone controlled release (40 mg) compared to oxymorphone immediate release (4.times.10 mg) under both fasted and fed conditions, and to determine the effect of food on the bioavailability of oxymorphone from the controlled release formulation, oxymorphone CR, and from the immediate release formulation, oxymorphone IR.

This study had a single-center, open-label, analytically blinded, randomized, four-way crossover design. Subjects randomized to Treatment 5A and Treatment 5C, as described below, were in a fasted state following a 10-hour overnight fast. Subjects randomized to Treatment 5B and Treatment 5D, as described below, were in the fed state, having had a high fat meal, completed ten minutes prior to dosing. There was a 14-day washout interval between the four dose administrations. The subjects were confined to the clinic during each study period. Subject assigned to receive Treatment 5A and Treatment 5B were discharged from the clinic on Day 3 following the 48-hour procedures, and subjects assigned to receive Treatment 5C and Treatment 5D were discharged from the clinic on Day 2 following the 36-hour procedures. On Day 1 of each study period the subjects received one of four treatments:

Treatments 5A and 5B: Oxymorphone controlled release 40 mg tablets from Table 2. Subjects randomized to Treatment 5A received a single oral dose of one 40 mg oxymorphone controlled release tablet taken with 240 ml of water after a 10-hour fasting period. Subjects randomized to Treatment 5B received a single oral dose of one 40 mg oxymorphone controlled release tablet taken with 240 ml of water 10 minutes after a standardized high fat meal.

Treatments 5C and 5D: Immediate release tablet (IR) 4.times.10 mg Oxymorphone. Subjects randomized to Treatment 5C received a single oral dose of 4.times.10 mg oxymorphone IR tablet taken with 240 ml of water after a 10-hour fasting period. Subjects randomized to Treatment 5D received a single oral dose of 4.times.10 mg oxymorphone IR tablet taken with 240 ml of water 10 minutes after a standardized high-fat meal.

A total of 28 male subjects were enrolled in the study, and 25 subjects completed the study. A total of 28 subjects received at least one treatment. Only subjects who completed all 4 treatments were included in the summary statistics and statistical analysis.

Blood samples (7 ml) were collected during each study period at the 0 hour (predose), and at 0.25, 0.5, 0.75, 1.0, 1.5, 2, 3, 4, 5, 6, 8, 10, 12, 24, 36, 48, 60, and 72 hours post-dose (19 samples) for subjects randomized to all Treatments.

The mean oxymorphone plasma concentration versus time is presented in Table 22. The arithmetic means of the plasma oxymorphone pharmacokinetic parameters and the statistics for all Treatments are summarized in Table 23.

TABLE 22
Mean Plasma Concentration vs. Time (ng/ml)

| | Treatment | Treatment | Treatment | Treatment |
|---|---|---|---|---|
| Time (hr) | 5A | 5B | 5C | 5D |
| 0 | 0.00 | 0.00 | 0.00 | 0.00 |
| 0.25 | 0.47 | 0.22 | 3.34 | 1.79 |
| 0.50 | 1.68 | 0.97 | 7.28 | 6.59 |
| 0.75 | 1.92 | 1.90 | 6.60 | 9.49 |
| 1 | 2.09 | 2.61 | 6.03 | 9.91 |
| 1.5 | 2.18 | 3.48 | 4.67 | 8.76 |
| 2 | 2.18 | 3.65 | 3.68 | 7.29 |
| 3 | 2.00 | 2.86 | 2.34 | 4.93 |
| 4 | 1.78 | 2.45 | 1.65 | 3.11 |
| 5 | 1.86 | 2.37 | 1.48 | 2.19 |
| 6 | 1.67 | 2.02 | 1.28 | 1.71 |
| 8 | 1.25 | 1.46 | 0.92 | 1.28 |
| 10 | 1.11 | 1.17 | 0.78 | 1.09 |
| 12 | 1.34 | 1.21 | 1.04 | 1.24 |
| 24 | 0.55 | 0.47 | 0.40 | 0.44 |
| 36 | 0.21 | 0.20 | 0.16 | 0.18 |
| 48 | 0.06 | 0.05 | 0.04 | 0.05 |
| 60 | 0.03 | 0.01 | 0.01 | 0.01 |
| 72 | 0.00 | 0.00 | 0.00 | 0.00 |

TABLE 23
Pharmacokinetic Parameters of Plasma Oxymorphone for Study 5

| | Treatment | | Treatment | | Treatment | | Treatment | |
|---|---|---|---|---|---|---|---|---|
| | 5A | | 5B | | 5C | | 5D | |
| | Mean | SD | Mean | SD | Mean | SD | Mean | SD |
| Cₘₐₓ | 2.79 | 0.84 | 4.25 | 1.21 | 9.07 | 4.09 | 12.09 | 5.42 |
| Tₘₐₓ | 2.26 | 2.52 | 1.96 | 1.06 | 0.69 | 0.43 | 1.19 | 0.62 |
| AUC₍ₒ₋ₜ₎ | 35.70 | 10.58 | 38.20 | 11.04 | 36.00 | 12.52 | 51.35 | 20.20 |
| AUC_{(o-inf)} | 40.62 | 11.38 | 41.17 | 10.46 | 39.04 | 12.44 | 54.10 | 20.26 |
| T_{1/2el} | 12.17 | 7.57 | 10.46 | 5.45 | 11.65 | 6.18 | 9.58 | 3.63 |

The relative bioavailability calculations are summarized in Tables 24 and 25.

TABLE 24
Relative Bioavailability Determination Based on AUC_{(o-inf)}

| Fᵣₑₗ (5D vs. 5C) | Fᵣₑₗ (5B vs. 5A) |
|---|---|
| 1.3775 | 1.0220 |

TABLE 25
Relative bioavailability Determination Based on AUC₍ₒ₋₂₄₎

| Fᵣₑₗ (5D vs. 5C) | Fᵣₑₗ (5B vs. 5A) |
|---|---|
| 1.4681 | 1.0989 |

The mean 6-OH oxymorphone plasma concentration versus time is presented in Table 26.

TABLE 26
Mean Plasma Concentration vs. Time (ng/ml)
6-Hydroxyoxymorphone

| | Treatment | Treatment | Treatment | Treatment |
|---|---|---|---|---|
| Time (hr) | 5A | 5B | 5C | 5D |
| 0 | 0.00 | 0.00 | 0.00 | 0.00 |
| 0.25 | 0.27 | 0.05 | 2.36 | 0.50 |
| 0.50 | 1.32 | 0.31 | 5.35 | 1.98 |
| 0.75 | 1.37 | 0.59 | 4.53 | 2.97 |
| 1 | 1.44 | 0.82 | 3.81 | 2.87 |
| 1.5 | 1.46 | 1.09 | 2.93 | 2.58 |
| 2 | 1.46 | 1.28 | 2.37 | 2.29 |
| 3 | 1.39 | 1.14 | 1.69 | 1.72 |
| 4 | 1.25 | 1.14 | 1.33 | 1.26 |
| 5 | 1.02 | 1.00 | 1.14 | 1.01 |
| 6 | 0.93 | 0.86 | 0.94 | 0.86 |
| 8 | 0.69 | 0.72 | 0.73 | 0.77 |
| 10 | 0.68 | 0.67 | 0.66 | 0.75 |
| 12 | 0.74 | 0.66 | 0.70 | 0.77 |
| 24 | 0.55 | 0.52 | 0.54 | 0.61 |
| 36 | 0.23 | 0.30 | 0.28 | 0.27 |
| 48 | 0.18 | 0.20 | 0.20 | 0.19 |
| 60 | 0.09 | 0.10 | 0.09 | 0.09 |
| 72 | 0.06 | 0.06 | 0.04 | 0.05 |

TABLE 27
Pharmacokinetic Parameters of Plasma
6-Hydroxyoxymorphone for Study 5

| | Treatment | | Treatment | | Treatment | | Treatment | |
|---|---|---|---|---|---|---|---|---|
| | 5A | | 5B | | 5C | | 5D | |
| | Mean | SD | Mean | SD | Mean | SD | Mean | SD |
| Cₘₐₓ | 1.88 | 0.69 | 1.59 | 0.63 | 6.41 | 3.61 | 3.79 | 1.49 |
| Tₘₐₓ | 1.48 | 1.18 | 2.73 | 1.27 | 0.73 | 0.47 | 1.18 | 0.74 |
| AUC₍ₒ₋ₜ₎ | 28.22 | 10.81 | 26.95 | 11.39 | 33.75 | 10.29 | 32.63 | 13.32 |
| AUC_{(o-inf)} | 33.15 | 11.25 | 32.98 | 10.68 | 37.63 | 17.01 | 36.54 | 13.79 |
| T_{1/2el} | 17.08 | 7.45 | 21.92 | 8.41 | 16.01 | 6.68 | 16.21 | 7.42 |

### Example 5

### BACKGROUND

A general dictum of opioid therapy states that ethanol should not be consumed at any time while receiving opioid therapy because of the known additive pharmacodynamic effects of ethanol and opioids (CNS and respiratory depression). Recently, however, information (both in vitro and in vivo) has emerged with some products (Palladone® and Avinza®) indicating that direct exposure to ethanol causes a disintegration of the ER formulations, which results in the rapid release of drug.

In July 2005, the FDA issued an Alert for Healthcare Professionals concerning hydromorphone hydrochloride (HCl) extended-release capsules (marketed as Palladone®, Purdue Pharma LP). The manufacturer of Palladone® conducted a clinical pharmacokinetic study to determine the effect of co-ingestion of graded concentrations of ethanol with Palladone®. Pharmacokinetic data from the study indicated that the co-ingestion of Palladone® and ethanol resulted in potentially dangerous increases in the peak plasma concentrations of hydromorphone. The average peak blood levels of hydromorphone were 6-fold higher (range of 1 to 16-fold higher) when Palladone® was taken with 240 mL of a 40% ethanol solution than when taken without ethanol. Lesser effects were observed when Palladone® was taken with 20% and 4% ethanol, 2-fold higher (range 1 to 6-fold higher) and unchanged (range 1 to 2-fold higher), respectively. These data coupled with in vitro data indicated that exposure to ethanol resulted in disintegration of the ER formulation, i.e., a formulation/ethanol interaction. Subsequently, the FDA tested another ER formulation opioid (Avinza®) using in vitro methodology. A similar finding was elucidated; this finding lead to changes in the labeling for Avinza® (Oct. 18, 2005).

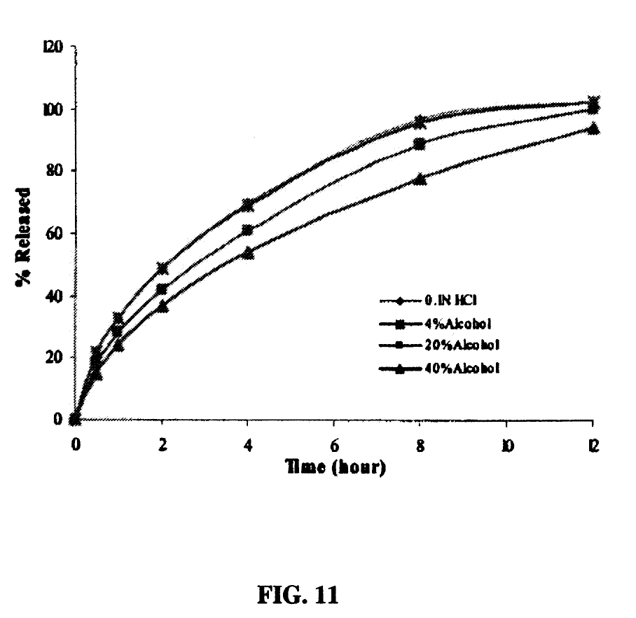

In view of these developments, Endo Pharmaceuticals, Inc. (sponsor) conducted in vitro and in vivo studies to determine if co-administration of its oxymorphone ER tablets with ethanol would be associated with a similar ethanol/formulation interaction. In vitro dissolution testing showed ethanol concentrations <40% did not increase the release rate of oxymorphone from the 40 mg ER tablet. The data indicated that, if anything, the release rate correlated inversely with the concentration of ethanol in the dissolution medium (see FIG. 11). These results do not support an ethanol/formulation interaction as had been seen with Palladone® and Avinza®.

To confirm that the in vitro findings were a predictor of in vivo activity, the sponsor conducted a healthy volunteer study that replicated the in vivo study conducted by Purdue Pharma LP. This study challenged the oxymorphone ER formulation using graded concentrations of ethanol. The lowest concentration (240 mL of 4% solution) was similar to modest alcohol consumption. The highest concentration (240 mL of 40% solution) mimicked a situation of serious and extreme alcohol abuse. The results of this study demonstrated that with graded concentrations of ethanol, there was no evidence of disintegration or dose dumping; but there was evidence of a “dose response” for the peak average oxymorphone plasma concentration. Increases in peak average oxymorphone plasma concentrations (Cₘₐₓ) of 70%, 31%, and 7% were noted with co-administration of 240 mL 40% ethanol, 20% ethanol and 4% ethanol treatments, respectively, compared to the 0% ethanol treatment. Some subjects demonstrated higher peak concentrations, up to 3.7 times higher, with 40% ethanol than in the absence of ethanol. The analyses of AUC_{0-inf} and AUC₀₋ₜ showed that there was no effect of treatment on these parameters (both p>0.05). For the geometric mean ratios (GMR) of 40% ethanol, 20% ethanol and 4% ethanol to 0% ethanol for AUC_{0-inf} if and AUC₀₋ₜ, the 90% confidence intervals (CI) ranged from 0.93 to 1.24.

This finding is analogous to what has been observed when oxymorphone ER tablets, immediate-release (IR) tablets, and oxymorphone solution are taken after a high-fat meal. Two earlier studies evaluated the effects of food on the bioavailability of a solution and tablet under fasted and fed conditions. In the first of those studies, the bioavailability of oxymorphone oral solution (10 mg) was evaluated under fed and fasted conditions. Both rate and extent of oxymorphone absorption from the oxymorphone oral solution were affected by food; Cₘₐₓ and AUC values were increased approximately 50% and 30%, respectively. In the second study, the bioavailability of 40 mg oxymorphone ER and IR (4×10 mg) were evaluated under fasted and fed conditions. The results showed that Cₘₐₓ was increased in the presence of food for both the ER (51%) and the IR (38%) tablets and AUC was increased by food for the IR tablets.

1. In Vitro Dissolution Study

In order to evaluate the drug release profiles in the presence of ethanol, an in-vitro dissolution study was conducted on 40 mg oxymorphone HCl ER tablets. The 40 mg strength is the highest strength of the product line, i.e., 5 mg, 10 mg, 20 mg and 40 mg, and contains the same amount of excipients, i.e., only the amount of oxymorphone in the tablet changes with dose. (Tablet color is the only formulation difference among oxymorphone dose strengths.) Therefore, we elected to test the 40 mg strength because it represents the worst potential of safety risk should dose dumping occur. The dissolutions were performed in 500 mL of 0.1N HCl and ethanol/0.1N HCl solutions at 4%, 20% and 40% ethanol concentrations, and the dissolution samples were determined by an HPLC method.

The tablets were intact throughout the dissolution tests in all media. The mean dissolution results are listed in Table 28. The profiles are presented in FIG. 11. The similarity factors (f₂) are calculated for the ethanol dissolution media against the 0.1N HCl medium, and the results listed in Table 29. The data indicate that the drug release rate correlated inversely with the amount of ethanol in the dissolution medium. Increasing the ethanol content moderately decreased the drug release rate. The similarity factors relative to the 0.1N HCl medium are 97, 60 and 45 for the 4%, 20% and 40% ethanol solutions, respectively. This indicates similar drug releases in 4% to 20% ethanol as compared to 0% ethanol medium, and slower drug release in 40% ethanol. The study indicates that the oxymorphone formulation drug release matrix is not defeated by high concentrations of ethanol and therefore dose dumping is not anticipated in the presence of ethanol.

TABLE 28
Means of Dissolution Profile of 540 mg Oxymorphone
HCl ER Tablets in 0.1N
HCl and Ethanol Solutions

| Time Point | Mean % Released (n = 12) | | | | | | |
|---|---|---|---|---|---|---|---|
| (hr) | 0 | 0.5 | 1 | 2 | 4 | 8 | 12 |
| 0.1N HCl | 0 | 22 | 33 | 49 | 70 | 97 | 102 |
| % RSD | 0 | 3.2 | 2.7 | 1.8 | 1.0 | 0.6 | 0.6 |
| Range | 0 | 21-23 | 32-35 | 48-50 | 69-71 | 96-97 | 101-102 |
| 4% Et0H | 0 | 22 | 33 | 49 | 69 | 96 | 102 |
| % RSD | 0 | 3.3 | 3.0 | 2.5 | 2.0 | 1.6 | 1.8 |
| Range | 0 | 21-23 | 31-34 | 46-50 | 66-70 | 93-99 | 99-106 |
| 20% EtOH | 0 | 18 | 28 | 42 | 61 | 89 | 100 |
| % RSD | 0 | 2.1 | 2.4 | 2.5 | 2.9 | 2.0 | 1.9 |
| Range | 0 | 17-18 | 27-29 | 40-45 | 59-66 | 86-93 | 97-103 |
| 40% EtOH | 0 | 15 | 24 | 37 | 54 | 78 | 94 |
| % RSD | 0 | 6.0 | 2.2 | 1.8 | 1.9 | 2.3 | 3.2 |
| Range | 0 | 14-18 | 23-25 | 35-38 | 52-56 | 74-81 | 90-101 |

TABLE 29
Similarity Factor (f₂) for Dissolution Profiles of 40 mg Oxymorphone
HCl ER Tablets in 0.1N HCl and Ethanol Solutions

| | f₂ | | |
|---|---|---|---|
| Medium | 4% Ethanol | 20% Ethanol | 40% Ethanol |
| Rel. to 0.1N HCl | 97 | 60 | 45 |

2. In Vivo Studies

This study was conducted in healthy volunteers to assess the pharmacokinetics of oxymorphone 40 mg ER tablets when co-administered with 240 mL of 40%, 20%, 4% and 0% (water) ethanol. The oxymorphone ER tablets administered were according to the now-available commercial formula of Opana® 40 mg strength, which also contains the inactive ingredients hypromellose, iron oxide black, methylparaben, propylene glycol, silicified microcrystalline cellulose, sodium stearyl fumarate, TIMERx®-N, titanium dioxide and triacetin.

2.1 Design

The study design was a randomized, open-label, single-dose, four-period crossover in 28 subjects. To block the opioid effects of oxymorphone, naltrexone HCl (50 mg) was administered approximately 12 and 2 hours prior to each oxymorphone administration, and again at 12 hours after administration. Subjects were fasted overnight for at least 8 hours prior to dosing. Water was allowed ad lib except from 1 hour before dosing until 1 hour after dosing. A standardized meal was served 4 hours and 10 hours after dosing.

Oxymorphone 40 mg ER tablets were administered on four separate occasions with 240 mL of:

A) 40% ethanol
B) 20% ethanol
C) 4% ethanol
D) 0% ethanol

Serial blood samples were obtained from 0 to 48 hours after dosing. Plasma samples were assayed for oxymorphone. Pharmacokinetic parameters for oxymorphone were determined using non-compartmental methods. Point estimates and 90% CIs for natural logarithmic transformed Cₘₐₓ, AUC₀₋ₜ, and AUC_{0-inf} were calculated using LSMeans. Any treatment in which a subject vomited during the dosing interval (0-12 hours) was excluded from the primary pharmacokinetic analysis.

2.2 Pharmacokinetic Results

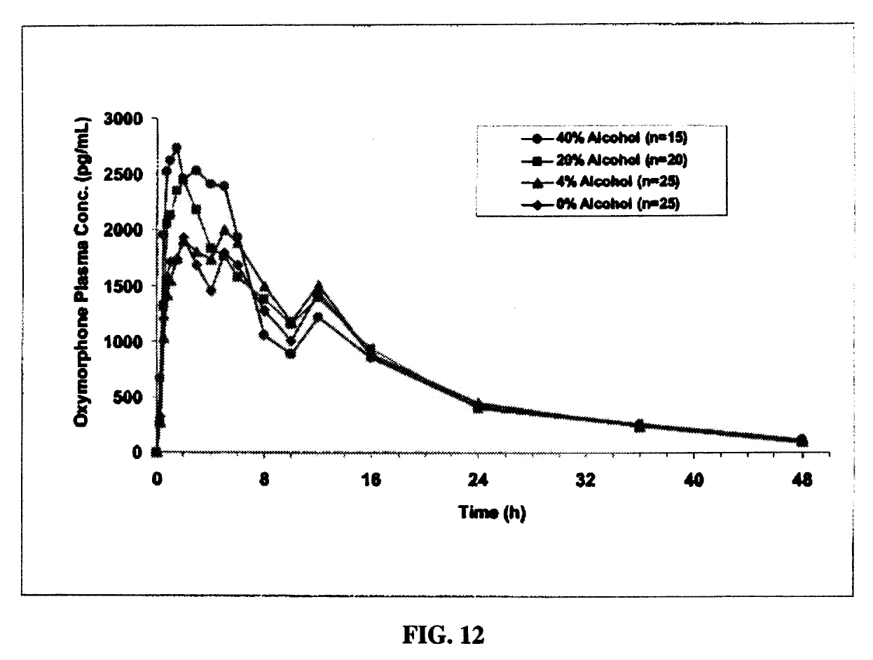

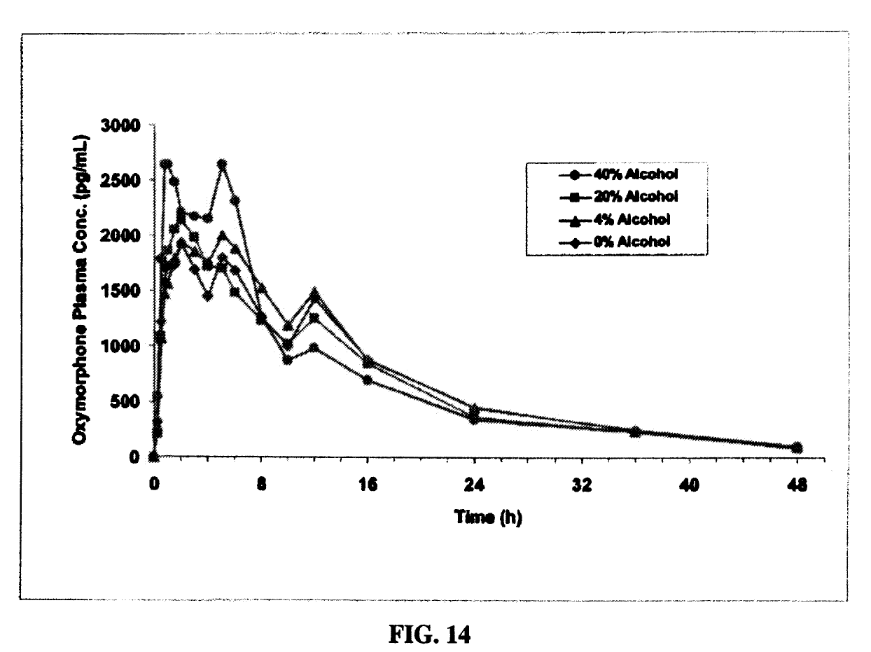

Thirty subjects were enrolled in the study. Twenty-five subjects completed the study, meaning these subjects received all four treatments. The protocol specified that subjects who vomited within the dosing interval (0-12 hours) were to have that treatment excluded from the pharmacokinetic analysis. There were 10 subjects who vomited between 0-12 hours on treatment A (40% ethanol) and 5 subjects who vomited between 0-12 hours on treatment B (20%) ethanol. There were no subjects who vomited on treatments C (4% ethanol) or D (0% ethanol). Mean plasma concentration-time curves for each treatment, excluding subject data from a treatment if the subject vomited, are depicted in FIG. 12. The protocol-specified statistical analyses of the pharmacokinetic parameters are presented in Table 30. Table 31 contains the geometric mean ratio (GMR) and 90% CI for those treatments in which subjects completed the study without vomiting between 0-12 hours. Although not the primary analysis, analyses of all subjects regardless of whether they vomited are presented in Table 32 (mean parameters) and Table 33 (GMRs and 90% CIs). Mean plasma concentration-time curves for each treatment, without any exclusions for vomiting, are depicted in FIG. 14.

The mean plasma concentration-time profiles in FIG. 12 show that the 40% and 20% ethanol treatments produce higher plasma concentrations during the first 4 to 6 hours compared to the 0% ethanol treatment. The 4% ethanol treatment mean plasma concentrations are similar to those for the 0% ethanol treatment. All curves are superimposable from 16 to 48 hours after dosing. Secondary peaks are seen at 5 hours for the 4% and 0% ethanol treatments and 12 hours for all four treatments. Although the 40% ethanol treatment was higher from 0.5 to 6 hours, the levels then declined rapidly and were lower than the other 3 treatments at 8 to 12 hours.

Cₘₐₓ was the only pharmacokinetic parameter that appeared to be directly related to the ethanol treatment (Table 30). From the GMRs (Table 31) it can be seen that the increases in Cₘₐₓ were about 70%, about 31%, and about 7% for the 40% ethanol, 20% ethanol and 4% ethanol treatments, respectively, compared to the 0% ethanol treatment. Changes in AUC₀₋ₜ and AUC_{0-inf} ranged from 1% to 13% for the ethanol treatments compared to 0% ethanol (Table 31). Other than Cₘₐₓ, there were no significant treatment differences for the pharmacokinetic parameters.

TABLE 30
Mean (SD) Pharmacokinetic Parameters by Treatment Per Protocol
(excluding subjects that vomited)

| | Ethanol Treatment | | | |
|---|---|---|---|---|
| Parameter | 40% | 20% | 4% | 0% |
| Cₘₐₓ, pg/mL | 3917 | 3089 | 2564 | 2373 |
| | (1672) | (1150) | (1037) | (870) |
| Tₘₐₓ, h | 1.50 | 1.50 | 3.0 | 2.0 |
| | (0.75-6.0) | (0.75-8.0) | (1.0-12.0) | (0.5-12.0) |
| AUC₀₋ₜ, pg · h/mL | 36385 | 35389 | 35146 | 33350 |
| | (12441) | (11495) | (12534) | (11864) |
| AUC_{0-inf}, pg · h/mL | 39973^{a} | 36889 | 37551^{b} | 36034^{b} |
| | (13595) | (12356) | (13452) | (11388) |
| t½, h | 11.3^{a} | 9.9 | 10.4^{b} | 10.7^{b} |
| | (3.5) | (3.2) | (4.1) | (4.7) |
| N | 15 | 20 | 25 | 25 |

Median and range reported for Tmax
^{a}n = 13
^{b}n = 24

TABLE 31
Geometric Mean Ratios and 90% CIs by Treatment
Compared to 0% Ethanol Per
Protocol Analysis (excluding subjects that vomited)

| | 40%/0% | | 20%/0% | | 4%/0% | |
|---|---|---|---|---|---|---|
| Parameter | Ratio | 90% CI | Ratio | 90% CI | Ratio | 90% CI |
| Cₘₐₓ | 1.703 | 1.476, 1.966 | 1.309 | 1.151, 1.488 | 1.073 | 0.952, |
| | | | | | | 1.209 |
| AUC₀₋ₜ | 1.129 | 1.03, 1.24 | 1.040 | 0.95, 1.13 | 1.055 | 0.97, 1.14 |
| AUC_{0-inf} | 1.127 | 1.03, 1.24 | 1.010 | 0.93, 1.09 | 1.022 | 0.95, 1.10 |

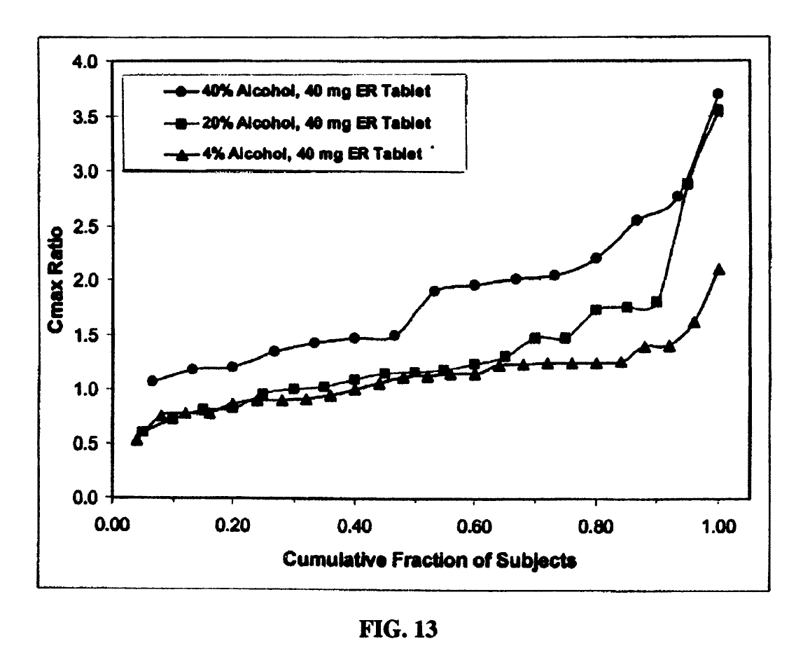

Individual Cₘₐₓ ratios for each treatment versus 0% ethanol are plotted in FIG. 13. These ratios are shown as the cumulative fraction of subjects within a treatment. For example, one-half of the subjects have a Cₘₐₓ ratio below 1.5, and 0.8 (80%) have a Cₘₐₓ ratio below 2.2 for the 40% ethanol treatment. The 40% ethanol treatment is consistently higher than the other two treatments. Overall, the Cₘₐₓ ratios for all three alcohol treatments have considerable overlap, ranging from 1.06 (about 5% increase) to 3.70 (about 270% increase) for the 40% treatment, 0.60 (about 40% decrease) to 3.55 (about 260% increase) for the 20% treatment, and 0.53 (about 50% decrease) to 2.10 (about 110% increase) for the 4% alcohol treatment. The 40% alcohol treatment differs from the other two treatments by a small (approximately 0.5 units) but fairly consistent difference, except for the cumulative fraction above 0.90 where the 20% alcohol treatment is similar. The 20% alcohol treatment Cₘₐₓ ratios are nearly identical with the 4% alcohol ratios for the cumulative fraction below 0.65. Again, only in a small fraction of the subjects (cumulative fractions >0.90) are there any appreciable differences between the 20% and 4% alcohol treatments.

For example, the Cₘₐₓ ratio for the 40% treatment can be an increase of about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200%, about 205%, about 210%, about 215%, about 220%, about 225%, about 230%, about 235%, about 240%, about 245%, about 250%, about 255%, about 260%, about 265%, or about 270%.

For example, the Cₘₐₓ ratio for the 20% treatment can be from a decrease of about 40%, about 35%, about 30%, about 25%, about 20%, about 15%, about 10%, about 5%, 0, or an increase of about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200%, about 205%, about 210%, about 215%, about 220%, about 225%, about 230%, about 235%, about 240%, about 245%, about 250%, about 255%, or about 260%.

For example, the Cₘₐₓ ratio for the 4% treatment can be from a decrease of about 50%, about 45%, about 40%, about 35%, about 30%, about 25%, about 20%, about 15%, about 10%, about 5%, 0, or an increase of about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, about 100%, about 105%, or about 110%.

The mean plasma concentration-time profiles without excluding treatments (n=25) in which subjects vomited (FIG. 14) shows the 40% ethanol treatment with a secondary peak at 5 hours whereas this was not clearly evident in FIG. 12 where only 15 subjects are represented. The 20% ethanol treatment (n=25) looks similar to FIG. 11 where there was 20 subjects. The 4% and 0% ethanol treatments have the same number of subjects (n=25) and are identical to those in FIG. 12.

As seen before in Table 30, Cₘₐₓ was the only pharmacokinetic parameter that appeared to be directly related to the ethanol treatment (Table 32). From the GMRs (Table 33) one can see that the increases in Cₘₐₓ were 62%, 15%, and 8% for the 40% ethanol, 20% ethanol and 4% ethanol treatments respectively, compared to the 0% ethanol treatment. Changes in AUC₀₋ₜ and AUC_{0-inf} ranged from −10% to 7% for the ethanol treatments compared to 0% ethanol (Table 33). The 40% and 20% Cₘₐₓ, AUC₀₋ₜ and AUC_{0-inf} increases were lower when subjects who vomited were included.

TABLE 32
Mean (SD) Pharmacokinetic Parameters by Treatment for All Subjects

| | Ethanol Treatment | | | |
|---|---|---|---|---|
| Parameter | 40% | 20% | 4% | 0% |
| Cₘₐₓ, pg/mL | 4124 | 2815 | 2564 | 2373 |
| | (2251) | (1227) | (1037) | (870) |
| Tₘₐₓ, h | 1.50 | 2.0 | 3.0 | 2.0 |
| | (0.75-6.0) | (0.75-8.0) | (1.0-12.0) | (0.5-12.0) |
| AUC₀₋ₜ, pg · h/mL | 33677 | 31815 | 35146 | 33350 |
| | (13772) | (13456) | (12533) | (11864) |
| AUC_{0-inf}, pg · h/mL | 37128^{a} | 34677^{b} | 37551 | 36034 |
| | (14803) | (13432) | (13452) | (11388) |
| t½, h | 11.7^{a} | 9.9^{b} | 10.4 | 10.7 |
| | (4.5) | (3.1) | (4.1) | (4.7) |
| N | 25 | 25 | 25 | 25 |

^{a}n = 22
^{b}n = 23

TABLE 33
Geometric Mean Ratios and 90% CIs by Treatment
Compared to 0% Ethanol for
All Subjects (N = 25)

| | 40%/0% | | 20%/0% | | 4%/0% | |
|---|---|---|---|---|---|---|
| Parameter | Ratio | 90% CI | Ratio | 90% CI | Ratio | 90% CI |
| Cₘₐₓ | 1.623 | 1.365, 1.931 | 1.145 | 0.963, 1.362 | 1.077 | 0.905, |
| | | | | | | 1.281 |
| AUC₀₋ₜ | 0.961 | 0.79, 1.18 | 0.897 | 0.73, 1.10 | 1.070 | 0.87, 1.31 |
| AUC_{0-inf} | 0.953 | 0.78, 1.16 | 0.920 | 0.75, 1.12 | 1.034 | 0.85, 1.26 |

3. Discussion

The in vitro study showed that 40% ethanol did not increase the dissolution rate of the oxymorphone ER 40 mg tablet. These data indicate that the formulation drug release matrix is not defeated by high ethanol concentrations and the premature release of oxymorphone in vivo when bathed in ethanol at concentrations up to 40% is not anticipated. However, the data from the human ethanol study (Table 30 and Table 31) demonstrated that co-administration of 240 mL of 40% ethanol, and to a lesser extent 20% ethanol, increased the Cₘₐₓ of oxymorphone from the 40 mg ER tablet while having no demonstrable effect on the AUC. The in vitro and in vivo results suggest that ethanol does not directly effect the formulation but has another effect(s) that can lead to an apparent increased rate of absorption of oxymorphone.

Interestingly, the increased rate of absorption of oxymorphone is also seen when oxymorphone 40 mg ER tablets are administered after a high-fat meal. The magnitude of the increase and the plasma concentration-time course are similar after both food and with ethanol. This observation suggests there may be a common mechanism underlying the increase in Cₘₐₓ.

The pharmacokinetic parameters measured following dosing of oxymorphone IR tablets and oral solutions were also affected when taken after a high-fat meal. In addition to an increase in Cₘₐₓ, the AUC for the IR tablets was also increased (˜40%), unlike the results for the ER tablets where AUC was not appreciably changed after ethanol or food. These differences suggest that the ER tablets are not releasing oxymorphone at an accelerated rate in the presence of ethanol, but that it is only the oxymorphone dissolved in the gastrointestinal tract that is affected by the food or ethanol.

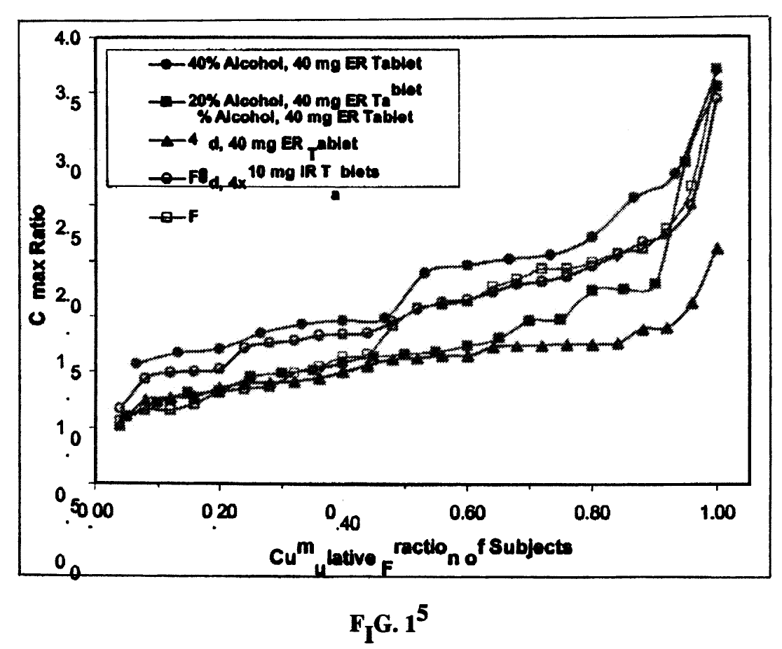

To gain further perspective on how the effects of ethanol and food compare, FIG. 13 was redrawn as FIG. 15 adding the cumulative Cₘₐₓ ratios taken from the effect of food study. FIG. 15 shows the Cₘₐₓ ratios with 40% ethanol are slightly higher than fed and 20% ethanol are slightly lower than fed. Overall the ratios suggest that large amounts of ethanol and high-fat meals have a similar effect on peak plasma concentrations.

As with all opioid therapy, ethanol consumption should be prohibited because of the known additive pharmacodynamic effects ascribed to these compounds (CNS depression). This effect is noted in various opioid product labels. The labeling previously submitted for the ER oxymorphone formulation has statements to this effect under “Warnings and Precautions.” Unlike the ethanol-formulation interaction noted for Palladone® and Avinza®, the in vitro results indicate no oxymorphone ER/formulation-ethanol interaction. The results from the ethanol study demonstrated that there is a pharmacokinetic interaction when oxymorphone ER 40 mg is consumed with 240 mL of 40% ethanol, which represents an excessive intake of ethanol, with resultant increases in peak plasma concentrations similar to those observed when oxymorphone ER tablets are taken after a standardized high-fat meal. The underlying mechanism of this phenomenon is not clear at present.

Based on evaluation of the in vitro and earlier iv vivo data, the increases in Cₘₐₓ seen in this study are not believed to be caused by early release of oxymorphone owing to disintegration of the ER delivery system (no dose dumping), but instead by an apparent increased rate of absorption, which is independent of formulation. It is believed that this “ethanol effect,” which is comparable to the “food effect” seen with certain drugs and dosage forms, has not previously been described for this class of drugs. Realistically, patients who use oxymorphone ER may drink socially or may inadvertently use alcohol-containing products concomitantly. Under these circumstances, it is believed that the situation can be managed with appropriate attention to patient safety. When the dosage form is misused and consumed simultaneously with large quantities of strong ethanol-containing products there may be an increased rate of absorption not unlike that seen with the food effect.

All references, including publications, patent applications, and patents, cited herein are hereby incorporated by reference to the same extent as if each reference there individually and specifically indicated to be incorporated by reference were set forth in its entirety herein.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the invention.

Alternative embodiments of the claimed invention are described herein, including the best mode known to the inventors for carrying out the claimed invention. Of these, variations of the disclosed embodiments will become apparent to those of ordinary skill in the art upon reading the foregoing disclosure. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the claimed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value. Thus, as a general matter, “about” or “approximately” broaden the numerical value. Also, the disclosure of ranges is intended as a continuous range including every value between the minimum and maximum values plus the broadening of the range afforded by the use of the term “about” or “approximately”. Thus, recitation of ranges of values herein are merely intended to serve as a shorthand method of referring individually to each separate value falling within the range, unless otherwise indicated herein, and each separate value is incorporated into the specification as if it were individually recited herein.

What is claimed is:

1. A method of using oxymorphone in the treatment of pain, comprising:
providing a patient with a therapeutically effective amount of oxymorphone in an oral dosage form; and
informing the patient or the patient's prescribing physician that the Cₘₐₓ of oxymorphone increased on average by about 70%, and up to about 270% in individual subjects, following concomitant administration with 240 mL of 40% ethanol.

2. The method of claim 1, wherein the oral dosage form is an extended release dosage form.

3. The method of claim 1, further comprising informing the patient or physician that the oxymorphone mean AUC after co-administration of 240 mL of 40% alcohol is not statistically significantly higher.

4. The method of claim 1, further comprising informing the patient or physician that the Cₘₐₓ of oxymorphone increased on average by about 31%, and up to about 260% in individual subjects, following concomitant administration with 240 mL of 20% ethanol.

5. The method of claim 1, further comprising informing the patient or physician that the Cₘₐₓ of oxymorphone increased on average by about 7%, and up to about 110% in individual subjects, following concomitant administration with 240 mL of 4% ethanol.

6. The method of claim 1, further comprising informing the patient or physician that the oxymorphone mean AUC after co-administration of 240 mL of 20% alcohol or 240 mL of 4% alcohol is essentially unaffected.

7. The method of claim 1, further comprising informing the patient or physician that the patient should not consume alcoholic beverages or medications containing alcohol when using the extended release oral dosage form of oxymorphone.

8. The method of claim 1, wherein the effect of the ethanol consumption on mean Cₘₐₓ of oxymorphone is about 40% greater than the effect of food consumption on mean Cₘₐₓ of oxymorphone.

9. The method of claim 4, wherein the effect of consumption of the 20% ethanol on mean Cₘₐₓ of oxymorphone is less than the effect of food consumption on mean Cₘₐₓ of oxymorphone.

10. The method of claim 8, further comprising informing the patient or physician of the effect of food on mean Cₘₐₓ of oxymorphone.

11. The method of claim 1, further comprising informing the patient or physician that patients should not combine oxymorphone with alcohol because additive effects may occur.

12. The method of claim 1, wherein the information is provided in the labeling information.

13. A method of using oxymorphone in the treatment of pain, comprising:
providing a patient with a therapeutically effective amount of oxymorphone in an oral dosage form;
informing the patient or the patient's prescribing physician that the Cₘₐₓ of oxymorphone increased on average by about 70%, and up to about 270% in individual subjects, following concomitant administration with 240 mL of 40% ethanol; and
informing the patient or physician that the patient should not consume alcoholic beverages or medications containing alcohol when using the extended release oral dosage form of oxymorphone.

14. The method of claim 13, wherein the information is provided in the labeling information.

15. The method of claim 13, further comprising informing the patient or physician that the oxymorphone mean AUC after co-administration of 240 mL of 40% alcohol is not statistically significantly higher.

16. The method of claim 13, further comprising informing the patient or physician that the Cₘₐₓ of oxymorphone increased on average by about 31%, and up to about 260% in individual subjects, following concomitant administration with 240 mL of 20% ethanol.

17. The method of claim 13, further comprising informing the patient or physician that the Cₘₐₓ of oxymorphone increased on average by about 7%, and up to about 110% in individual subjects, following concomitant administration with 240 mL of 4% ethanol.

18. The method of claim 13, further comprising informing the patient or physician that the oxymorphone mean AUC after co-administration of 240 mL of 20% alcohol or 240 mL of 4% alcohol is essentially unaffected.

19. The method of claim 13, further comprising informing the patient or physician that oxymorphone may be expected to have additive effects when used in conjunction with alcohol.

20. A method of using oxymorphone in the treatment of pain, comprising:
providing a patient with a therapeutically effective amount of oxymorphone in an oral dosage form;
informing the patient or the patient's prescribing physician that the Cₘₐₓ of oxymorphone increased on average by about 70%, and up to about 270% in individual subjects, following concomitant administration with 240 mL of 40% ethanol;
informing the patient or the patient's prescribing physician that the bioavailability of oxymorphone may be increased in patients with hepatic impairment; and
informing the patient or the patient's prescribing physician that the bioavailability of oxymorphone is increased in patients with renal impairment.
